SEQUENCE LISTING (37 sequences)

SEQ ID NO: 1 - DNA, 29158 bp, Homo sapiens
  source 1..29158: mol_type = genomic DNA
ctgcctgtgctgtgcctgcctcctagagctcattccctacgccccgactctgtcctggacagcgtgcccaccagccatggcggggccccggggcctcctcccactctgcctcctggccttctgcctggcaggcttcagcttcgtcagggggcaggtaagtgtgagccagtcgcaggggcacacggcgtctgggccctgccggccagcggtgggaacagggagggactgacatggaggggcaggggcctggggagaaaccgcagctggcgcctgtctggcctcttagcaggcctgggggctggctattgtgcccacctccccagtctgaggtctgcgtccagccacccagggttcaggggtgtgtcagcccaccctcctctgactatgcggggccggggctccccagcgtgcagctgaagtgtacagaggcaggcctgtcctgcccttctggcccagacggaggcccaggtcttatgtggccctcacctgggggttgaagatgtgatcacctccaacccccgaggactacagattcacgtggggcaagtgtgtgcacatgtgtgtgcctgtgtccagccccaagagtaccgggaagcagaatgagtcactcacccttagtgtccggggttatgagcagctggcaaggacacacttcagcctcagagctgccacagagcagccacagccccgggcaggtccccttgaatcctcacagcctcggggcaactcctcctgtcctcctcgtgaggaacccgaagccctaggatgatggggacaggccattcccactctaaagccatgctccaaacccctgcctgtgatggccatccgtgtgtgctggcgagcagacagggtcctggtatgcagccacagccagcccctcggtcaggtccctgtgacaggctcctggctgcaggtctgctcaggccccatggtaggggcctctgggagcttctggaagagcctcccaccctggtgatgggggtagtctctctcctgtgaggctgagcgtgtctgcctcctggagcagcaaccactatccccatggcacagatggccagactaaagtccagagcggggaggacttgcccaggatcccacaggcagtccagccaggggcctaggccccctgccaccatcaggcgctttctcttggatgcctgtggtccctcagagaggcagcagggagaagggcctcaccagcttcaggaccaggccagggctggggacaggaagatatgaggctgagaaggcgacagtgatgggggaagagccaagggttccagggctgtgctgagagccctgcaggtaagctcctgccctccccttaactcagtttccccctgtgtaccatgcaagggaatttcaaaggcctccgccacctcctcatgctctgggcagtgaggacagaggtcagtgaggggcctcagattcgagttttttccttgtcagagacctatggctctgaccactccttgtggatggggctgcagcagtgctggccactctaacacagctacgtggcctctatcccagcaccctgcatagccgagagccactgtaagtgcagttggccctctgtatctgtgcaccctgtacttgggttcaaccaaccctggattgaaaatattcaggaaataaaatggcatctctactgaacatgttcagactttttttcttttgaggtggagtctcgctctgtagcccaggctggagggcagtggcgtgatctcggctcagtgaaatctccacctcccaggttcaagcgattctcctgcctcagcctcccgagaactgggattacaggcgcccgccaccacacccagctaatttttgaatttttagtttcaccatgttgaccaggctggtctgtaactcctgacctcaggtaatccacctgcctcagcctcccaaagtgctgggattacaggtgtgagccaccgtgcccaaccccatgttcagacttttctatgtacatagcatttacattgtattcggtattataagtcgtctaaggatgacttaaggtatacgggaggatgtgcatagattatatgtgaattcgatgcctttttttttttttttttttttttgagatggtgtctctctctgttgtccgggctgcattacagtggcgctatctcagctcactgcaacctctgcctcccagcttcaagggattctcctgactcagcctcccaagtagctgggactacaggcgcacaccaccacacttggctaatttttgtgtttttattagagacagggtttctccatgttagccagcctggtctcgaactcctgatgtcaggtgatctaccctcttcggcctcccaaagtgctgggattacagatgtgagccaccactcccagcctcgattccattttataccagggacttgagcatccacagattttggtatctgaggagggtcctggaaccagtcccccatagacaccaaggaacaactatattaggagtggaagctctgaggggaaaggatatcccgggtaacctgttcagtaagggcagcaacaggactggagtgaagatgggtttgggcagcgaggcaggaaggctttcctggaaggcccttagcacttgcccattcagcagcttttcaccgagggcacgtggctaggtgtgtgcagtggagcctgtggcgggagaggagcacggtccagaacccagccctgcaccctccctgatctgaccaaaggtagggggaagcagtttggggttccttcctctctgacagggtttagattctgggctctcagccttaaaaagtccccatgaaccaggtcccagcttgggagaatttcatccaccttctcaccaaagacttcacacttcgtgtctcctcaacttcccccagcagcagctctgtgggaagcagacattgcaaaatcaggtctgcggggggtgtggggttgggggggagacagcacccgctctggctcagtcccgaggtgcccccaggaaggagggaatctgactcagcactcaggccgtgatccagcctcactccacccagctggaccaacggcaagatgtgggctcagaccgccgatgctccttccaggccatggggcggaggcagggcccctcggctgtggtctcggagaagcactgatgtggcagcaggcttaccattcatcagcatttcacacacgtttacaaacacccaggtcctgtgagtgagtgtccacaccaggccccaaagctcccgcttccttctgggccctaactcacccatcgctccatctagccctgactgtctcagagtgcgaccagcacagtcttactcagcctgagcccttatccagagctgaccaccattcctccaagagctggtccacagccagaatggtgaccgcgctggcagctttgcccagctggccaggtggctttgaggggagcccacctcctgccaagcctctaccctgagccagcagctgctcagccctcaagttacccaatttcctagtctctccctggctcagcaaaggcgagcctcaggatggggaacgagaccacaacatcccagctccgccgttcaacacaggctgctctgagcaacttccctgtttgcagaccgcagaggcatgagcggaaaaggccaaatcacggtagcacagaggctctggggccctggtatttgccataagaaagtgtcaggggcaccatttctccttttctcttgctttgagaagccacacatctgtaaattattccaggaaagggatgttctggggtcactccctttggaccacagtgtggtcagcatttttcaagatgagaacatctccacttctctgggccatgcccaaagcctcccaaacacctcccctccaacctatgccccttccctatgccccttcctcccactagaactgggagtttagcttcacgcctcctggttcagtgctgaaggttgctgggactttgttgccgtagaaggctggtggacttccaggagctatggcaggccagtgaatgggactccaggcctggggcttttgtcagcatccttgtgcccatgccgtggtggcaggctggactcctcacatcttccacgccccacccagttgctgagaccttctcccaattcatctcaccaccccaagccctctcaaatatctcccaatccatttccactgtccctgccttggcccaggccaccttcaggtaatgatggcctctttcccaccacacctgcccctgcgactgtattctccatgcagcagccagagggccaatttaaaacaaactggccggccgggcgcggtggctcacgcctgtaatcccagcactttgggaggccgaggcgggcggatcacgaggtcaggagatcgagaccaaccccgctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccatctcaaaaaaaaaaaaaaaaaaaaaaaaactggccaggcacagtggcacatgcctgtagtcccagctactcgggaggctgaggcaggaggatcgcctgagcccaggagttcaagattacagtgagctagggtcacaccactgcactccagccagggcgacacagcgagaccctttctcaaagacaaaaaaccataaactgagtcatggggctgccctggactctgcctcacccaggtttacggagctcctgcaccccagctgccagcccctctccagcctcccctcgggccactgcctgccttgtcacacatttgtttttacttctttccactcccggcctttgcttctgctgtcccctaggccaggagcactgtccttgaccaattctcatgcatccttcaggtcccaagtcaatgaccacctccctcaggaaaccacctggggccccagccaggatgccctgggtttcctcagaggctggcatggcccgttgtctcctgtgactgcccagcccaaacacttgcagaccagagtccctgcacacagctgctcacgtgagcccagcctctcagggccagggcccttagtctctgattcctgggcagcggagaaagcccttgcgcttggttagagagggtcatttggtagcaacgtgggaaaatgctcagagcagagtggtggctggaggtccagccccacggtgcatggctggagccggcatcgtgcctgtgcccatgagttgaggatggagtcgccgtgtcctcccaagacagcatcagctctgtcatttattgtttctttatctttaaacaaaaggcaaaagtgagcactaggctgggaatctgggtttagtccctggttcacaccctgttcatggatgagattgggtagcggccatgtcctgggcttcagttttctcacctgaaatgggcttgggggagcaacgtggctagattctctagtgtcagagggtgtggactcagagccttctgggccgggcacataaggactcgatgtgacattaacaacttgtaacagacaggtttcacagggccttccccatctgcactgcattcccactgagcgcccccagggcattctgtggacccccaaagaactcagctctgtgcccagaaccctctgcttactctgacccctgagcagatgggaagtggcagctggaggggtgtgagcccttgcagcactgaggacagggtcaggactgggcgtggggtccaggcccctgaccacaccctgcctgtggccccaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccggtgcgggccacccctgtccttgcctgtgtccaggagcatcctgtggggtggcagggactcggggagcctgacaggcccacaaaatgaggagaagcactcacttatccatgcccctgtggccccacctgggtggtggagataggcggggaggaaggggtaaaggcgaggggcaggaggggtttatggggcacctgctatgtgctaggccctgtgctcagctctgggccaggcagagcagggaggtgggtgggagccccggaagccaatgtggggaggctgggtgggggctgtgcaagggagccttttccatcaccctgcccaccctctgcccccagctacgaagtacagctggggggctctatggtgtccatgagcggctgcagacggaagtgccggaagcaagtggtgcagaaggcctgctgccctggctactggggttcccggtgccatggtatgggagaaagggggaccccgccttgcgcctcccacccagccccagctctgggcaagccctctgcaggagccacctgcattccccttcaacttgtttgtttaattaatactcaccgagcgcctccaaggctcggcccccttgctcttgcttccttgggggaggggccgagtctggggttggccagagctgctggggtcagggtaagcggagggttctgggcacagagctgagtactgtgggggtccacagaatgccctgggggcgctgagaccccatgcaatggccacgggacctgcttggatggcatggacaggaatgggacctgtgtgtgccaggtaagggctgggcaaggtggggtggaggctcagaggggccacgctgacttggtgcatccaccaccaggaaaacttccgcggctcagcctgccaggagtgccaagaccccaaccggttcgggcctgactgccaatcgggtgagtgcttaaaaggcaagagggcacggccagcgtccacctgggggcccacgcagatgcagtacctacaaccagctcccacctgtcccttcagcctctgtcccagcccctcagatttgcacatccctttcaggaacatgtgctttgttttatcacacctccagccctttgcacatgctactcccctgagaggccttctccaggaagccctctgtgagccccaggccgggccagtgccttctgtggtctcccaccacccctgtggtcccgcatcacagctctgtactcttaaggttgtacctgcccgagctgagttctaaaaatacagagagcagggacccgatgtcccagtgtgtgcctgggagaggctggggccccatctgtctctgtgtgtccctcccagtgtgcagctgtgtgcacggagtgtgcaaccatgggccacgtggggatggaagctgcctgtgctttgctggatacactggcccccactgtgatcaaggtgagcagcgccactgggatagcctagggcagcaggtccctgtggccagagcaaaagaggccgactgggtgaggatggggcctgagcctcagcaggacctccgcctggagcctagttggggaaggggtgtcatctgagacctccactgtccagaggcccagcaaggtcagggccctgctccaagtcacacagaacaggcaaggcccctttgcccctgtgttcctccccagcctgggcttgggctcatcagtgctctctccactctgcgcagagctgcccgtctgccaggagctgcgctgtccccagaacacccagtgctccgcagaggctcccagctgcaggtgcctgcccggctacacacagcagggcagtgaatgccgaggtgagcctggactcagaggccagggacttcagctcagggcgggcgggggctgggagagcatcctttaacccagcaagtgcaatgtaattcacaaggagggagagccattcctaaggggagcctatcctcaaggcctaggtcaacctgctggccccggccttcctggtgaaagctgtggcggagacagggctcctgggtgcttggctcacttgggctctctctctgccctggcagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggtgagccagggtcccaggccggaactgtccccacagtgcacccaaacactggctatgggaccctgggcaagtcacaggccttctggtagcctcagtttccccacttgtaaagtggggagaggataagggtcctcttcagggaggggctgagcagggctctatgggtagggcagacaccagtggttcttggggttggacgtggtgttcccctcctgccctgtcgggggccaggccttctgcacctgccggccaggcctggtcagcatcaacagcaacgcttctgcgggctgcttcgccttctgctcccccttctcctgcgaccggtctgccacttgccaggtgaccgctgatgggaagaccaggtgagcacaggtgcctgggagcagagacagtgggttgggcaagcgtgccctctgcgggaaggcgtggggggtgcggtggggggggcctcagcctccgccctgactggctctccctgggagcccagctgtgtgtgcagggaaagcgaggtgggggatgggcgtgcctgctacggacacctgctccacgaggtgcagaaggccacgcagacaggccgggtgttcctgcagctgagggtcgccgtggccatgatgggtgcgtgaccccactgcttgcccacgacccgacccctcacccccagcacagttggcagggagggaggagctgcctctgcagattccagtttctctgcccagggctgtgggaacaaacaggtggctgtggggggtgcatgcagggggtgggagctgctctccaacctcaaggcccccatctaccctaaatctagggagaatacccccaccccaactcccccacaccaggtctgatgcccgaacatcttctgagatccccagagtctccccagctcccttctgatgctccctcaccctgccccccagaccagggctgccgggaaatccttaccacagcgggccctttcaccgtgctggtgccatccgtctcctccttctcctccaggaccatgaatgtaagcccctccccatggtggagctggccactggccctcacctcctcccctggatgcatccccagtcttcagggccacctggaaggcatcagggcctgggatgcatcctgtcccctccattgctgggtagaggtggtgtgaagaggactggccccccggctcactttgctccccgccttgcgtctgcaggcatcccttgcccagcagctctgtagacagcacatcatcgcagggcagcacatcctggaggacacaaggacccaacaaacacgaaggtggtggacgctggccgggcaggagatcaccgtcacctttaaccaattcacggtgagggaggagcctcagcctggggacaagaggagtcacagcctggcaagggtgtgcaggtgggagggagcctccggacagggggctggcatatgggatctggccagacctcttgtgtctcaccagaaatactcctacaagtacaaagaccagccccagcagacgttcaacatctacaaggccaacaacatagcagctaatggcgtcttccacgtggtcactggcctgcggtggcaggccccctctgggacccctggggatcccaaggtgagccagcatcctcccctcccctcccctgtgctgctgggtaatctcagacccctgcagagagcagtgagtgggcaggagccattctactcagggctggagcgcagcttctccccgctgggctgaagcagcctcaccccttccctggggccctcacactcatccctccttacagagaactatcggacagatcctcgcctctaccgaggccttcagccgctttgaaaccatcctggaggtaagctcgggggaggggtcctagcccatgtgggcttctgggcttctgagccaggactcattatcccagattctgccccacagtgacctggggttctgaagtagcatggcctctgcccatgcccccgtcctcaagcctgaggttctgtggtagcatggacccattgcccatgtctcccccttactcagtgggagtttcctgcaggccaaaggtggcagggcccaggcaggactcagaggtggaggctggccatgcaaccccctgagcctcccttgcaccaccacagaactgtgggctgccctccatcctggacggacctgggcccttcacagtctttgccccaagcaatgaggctgtggacagcttgcgtgacggccgcctgatctacctcttcacagcggtaagctcagcgggagaaggggctgcgggtatgggggcaccaggacctccacgctcccccacagttcctccaggagccaaaccctctctctcccctgccagggtctctctaaactgcaggagttggtgcggtaccacatctacaaccacggccaggtgcgaggtctttttctggggggcggccagcttgtacccggtgggcctgcctgcaggttctggacttccccaaggccccatctcttctccatctcccttcccttctcatctgttctgctatagcacagagcctagggaccaagggggtggctgtgagcctcctgggcaggactgggcgtggccccaaggtatggccagagcccagcctaaagccacactgtcccttgccccagctgaccgttgagaagctcatctccaagggtcggatcctcaccatggcgaaccaggtcctggctgtgaacatttctgaggaggtgaggtgcacggacacctgggcggatggtggggctggcggtgaccagctgcccacttggcctctcagccttgtgtgctccgagccccgtggtgggcaggacactgctaggccatggcgggctcatggggaaggaagcaggcagagatcaaattcctctgctggggcccaatgagagttaagggggaggactccaaaaaggagcatggagcacacggtagggctgccccatgtggcagaagccatgtgggtaaggacccgcagcctggagcagtgtcgttggccagagctgaaggagaggtggctggtatggccagcggggccttcagcagctcactgagctgcctgtctgctctaactgggctgcaggggtggaggtgggcagccccaccgggccaaggccagccaggctgggggttggctcagtgggtctctgccgcaggggcgcatcctgctgggacccgagggggtcccgctgcagagggtagacgtgatggccgccaatggtgtgatccacatgctggacggcatcctgctgcccccgaccatcctgcccatcctgcccaagcactgcagcgaggagcagcacaagattgtggcggtgagcctcgcctgcacggccagggccctactcactaacccctgtcagcgctggagcggcaatcctcttcccagggagaggcgctaagtcagggctgggctgacgccggctctgaggccctgctggcagactcccaaggacccacctgtgcttcccccacgctcaccctcaccaggactatgattccatctttgagatttgttttgttttttgaggtagggtcttgctctgtcgcctaggctggagtgcagtggcacaatcccaactcactgtagcctccgcatcctgggttcaagcgattctcctgcctcagcctcccgagtagctgggattacaggtgtccgccaacatgcctggctaatttttgtatttttagtagagatgggatttcaccatgttggccaggctggtttcaaacctctgacctcaggtgatccgcccacctcggcctcccaaagtgctgggattacaggcatgagccactgagcctggcctgagataactcttgcatcctccaccgctccatcccctttcactcactctgtgcctgcctctggcctctctgctgttttccagagactctagctctgcccggctcggggcctctgcccctgctctcctctgcctggaatgccagtgctgggcttcctgtggttggctcttctcatcccttaatccccactcaatgttcccttctctgggtttgccgagtaaataaacaatgactaacccctctccccatcctgcccctgccctgcccagggctcctgtgtggactgccaagccctgaacaccagcacgtgtccccccaacagtgtgaagctggtgagcacaccttggcccagctctcagggcctcctgactgcctgttgaggtttctgccctgggtcacaggggtgggtgggggaagcagaggctgggaggctagatcacacctggaggctaagtgcttcgcagccccactggaccagggttagagttctggacccagggggcagaggtcccttaaccagggctgcctagcaaaggggctggcccagaacccacacccactgactggctttgcatggcccaccctaggacatcttccccaaggagtgtgtctacatccatgacccaacggggctcaatgtgctaaagaagggctgtgccagctactgcaaccaaaccatcatggtaagcgtgggcatgggtgcccactcccaggtcacctgggcacctgctgttcctcgggggcccccaagctggggctgagtgggctccctccctcagaggatgcactgcagcctgacggtggaaaaggggtcccagagaaaccttatccactccgtcactctggagatggggccagggagcagaggcagcctgggcagggagggcatggacccagatggcctcttggacctgttctgtctcttataggaacaaggctgctgcaaaggttttttcgggcctgactgcacgcagtgtcctgggggcttctccaacccctgctatggcaaaggcaatgtgagtcccatcctctcctggggtgaggtatggggaacacaaggactccttcaccggttggccagtgactggctgtgtgtctgggccaagcctgccactctggccttgggattctcatatctatgcagtggaaagttggatcaaattatttctcatattttaattaaaaataattagagaggccaggtgcaatgtctcatgcctgtaatcccagcactttgagacgctgaggcgggcagatcacttgaggtcaggagttcaagaccagcctggtcaacatggcaaaaccctgtctgtactaaaaatacaaaaattagcctggtgttatggcacacacctgtaatcccagctactcaggaggctgaggcaggaaaatcacttgaacctgggaggcggaggttgcagtgagccaagattgcaccactgcactccagcctgggcaacagagtgagattccatcccaaaataacaataataataataataataataatagatagatagatagattttttggcatttatttccaaaaaggctacttggtggttttcttttgtgttctgtttttaccactttgtaatatcacggttactttagcacctggctatagaagaacacaggagttatgtgtttctgcatccagcaaatgttgctcataaagcctccagggccaggtgccttttaggggctaggtctttggcggcatcagtgattttgaagatcccttccaggatgagctctcacgggtagcaagagctcagagccagcccatgaaaggagggggtgttgggagaggggatgtagagagtccctttcccatgactgatcctgccttctgctcactctctagtgcagtgatgggatccagggcaatggggcctgcctctgcttcccagactacaagggcatcgcctgccacatctgctcgaacccaaacaagcatggagagcaatgccaggaaggtgggtggtcctggctcaggccacctcctagggagaaaaaacgtctgcctaaagatgggtctgggggctctcaagaagggaaacgaagccccaggaggagggacgaaggccaaggggagtggaggaagatttgcctaaaaatgggtctgggggctctcaagaagggaaacgaagccccaggaggagggacgaaggccaaggggagtggaggaagatttgagaacagaaaggagatgctgtctgcttcacccaggccaggaagggaactgtggcttaaagtatgcgggacttaggtcagacacactgaagaacttcctgatggtggtgttgggatcagggcaactagaatgggataattgaaagctcagaatgcccccctcacatttcatctgcctgcccattgagtctgagataacctctgatttttccaccctgaagtcaaatctgtactggctgccccactccctatatcccccaaacccatcctggctcccagccagccctgcctcctgcttctcagtttccttgctcccatctactccatacagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccaggtgaggacccacaccttctgtctgccccacccgtgacctttcatacctgaggctcactggagccccctccttcacccacccccagatgtcgctgtcttgatggctttgagggtgatggcttctcctgcacacctagcaacccctgctcccacccggaccgtggaggctgctcagagaatgtcagtccccttgctccttccctgaagttctgacccagaggcaggaggtgggatgccctggccctctgcctacgttcttgtgtccctcagttatttatccatggctctcacaggctgagtgtgtccctgggtccctgggcacccaccactgcacatgccacaaaggctggagtggggatggccgcgtctgtgtggctattgacgagtgtgagctggacatgagaggtggctgccacaccgatgccctctgcagctatgtgggccccgggcaggtgaggtgcagcagagaaggggtgggggccttggttctgggggactctctccctgccccatctgactcctggaatgcagagtcaggtgctcagggtctggagcctcaggtgcagatgagaacccagtgctgacatgctgagtagtagcatctgggattcgctccttcatccattggctcattgctttcccaggtgttttcaggcacctattccatgctagtccgtgttctagatgtagggaatgtgatactgagcaaagcaggaaaagcccctgcccgtgggagtgacctccccaaaactttagcctcatggaaatccagccccggagctttgcagtgtctgaatctagtcacgggagctcggaacctggagccttccatgtggagcatttcaggactggtgaaccagtgtttggacaggcattgaagactgtctaacgcgccaagtgggaggccaggcctggctggtcagggtcagcctctgcatggccaggcctgtggggctccctgtcctcccaactccaaggtggcctctgggcaccagcacgtggggccttagcagtaagaactgacaggtgtaagagctggagctccaaaccccattagggccttaattcagaacagaggtgttgaatgagccagtgagatgctcccaacgtgcaaacaagggcacctgagagctgaggggtctcgctggtcactcacctgcagtgtagacaagcccagggacactggcatttggttgggggattagagcagctgagtagggtggcaggggtggaggtgggatctaagccactatcctggggctggccttggcaggtacctacacacttgccacaagctgttggcctcaatggccctggcagggccccaggctccaagtgcaggagcaccaagtctccacggatgcttctctgcaaatgctctcacctccgtggactcatcctgagagccagtctccccttagggcctctgtctccccatctggagcacagatctctgagaggagggctgggagaagttcctctggggtctgggcagacctccctgggggcagtgaccagagagagccaagtaatggaggaagaaagggttggatgctcatcatgagacaaggccgtctgttcctaacctttccagagccgatgcacctgcaagctgggctttgccggggatggctaccagtgcagccccatcgacccctgccgggcaggcaatggcggctgccacggcctggtaagggggtgcaaggctcagagccctggggaagctttctggggtgagcctgggctgcagctctctccctccctccccatctctgggtgtctctctgtaccccctcaggattaagatctttctaaacttcaaatccaaccatgtcactcctctcccccttcacccgcccctgcccagcctcgtctctctgttccactcctgccccacttccactgtccttggctacgctgagctcctcaccatcccctgcccttgctccactgtaccacactcctgcttctgcaagctgcacctgggcctggctagctcctcctgctctcagctctggctcctggggatacgggcctcctgggtcctcccacagccctgagctccctcgcatccacctggcatgggtaatgcttgtgtcaaatgcccagtgtcctggagagcagggctaggcacaccgaaggagtgccaccaacacagactgggtggctgcagagcaaggggcaggggctggggctgggtggctggacaggcagaaagatgggggagggaggggcccagctgccatctggttctgaatggaggccctttctcactcccaccccaggccacctgccgggcagtggggggaggtcagcgggtctgcacgtgcccccctggctttgggggtgatggcttcagctgttatggagacatcttccgggtaaggggtgctcagactcgttttctgtcttccccgtgcttgggaaggagccctctccagcacctcaggtgggaaaggggcactgctcaggatccatggtggggaaggggcttgaatttgcccctggtcctgtgacctcagacttttcccttcccttaggagctggaggcaaatgcccacttctccatcttctaccaatggcttaaggtaggacagggcagaatgctggggttgagggctcaaatccaggaggcctgcctggatggaggggtgccatataacatctccaagcgtgggaggttgggggcagggctggagcctagagcaggggatctgaagatgatggctgccttcctcgctcctcccgcccctgctccctgtgtgcgtgcagagtgccggcatcacgcttcctgccgaccgccgagtcacagccctggtgccctccgaggctgcagtccgtcagctgagccccgaggaccgagctttctggctgcagccaaggacgctgccgaacctggtcaggtggggccgccattgccaggctgtgggaggggcttccttgagggacccagtccctccccagcgagtcctcagcctggcaggcactcaccccttggggtgggcccagccccaaaggtctctgtaagttacactaacctgattccatgacccccctaaactgtgccctgtcagggcccattttctccagggtgccctcttcgaggaggagctggcccggctgggtgggcaggaagtggccaccctgaaccccaccacacgctgggagattcgcaacattagtggggtatgtggtgaactctgggcagaaaaggggaccaggtagggcatgggagggagcctgagtggctgcaggctctcccacatcaggggccccatgggatgagattggggtcccctcgccctgcccagagccgggctcaaaggatgtgctcaggacgcattgctgatggacactgagtcgctgggccaggagtgactgcctgcaggggctctctgtccatctcagtctgcacatgcacacccgcccctgtcaccacgggccctggggagggtctgtgtgcccagctggtgggtcctgagtctgactcaggatcagagtcagctcctgggaggagcttctgtgcagaacccagcttgtgggccagcctgcggaccacctgtgggtgcctgtgtgcctggcagtctctctgttggggctgccccacctttaagggtctatctgtgcccacctgtggctaagctctgctgctgccttccagagggtctgggtgcagaatgccagcgtggatgtggctgacctccttgccaccaacggtgtcctacacatcctcagccaggtacagcaggaggagggtgtgtgcagggaaacttgcaggcaggccgtgaagcaatgacatactgaccaggccctgtgctctgtaccaggtcttactgcccccccgaggggatgtgcccggtgggcaggggttgctgcagcagctggacttggtgcctgccttcagcctcttccgggaattgctgcaggtacggaaggctggcaagaggggatgtgcctgctcgggggacactgtgccccaggtccagagggacctggctggctcccagtgtcaggactgacaactaatatgcccatccctgacctccaccccatccctagcaccatgggttggtgccccagattgaggctgccactgcctacaccatctttgtgcccaccaaccgctccctggaggcccagggcaacagcagtcacctggtgaggccgtggggatggggcaatggcagggagggcaaaggcatagagggcgagcctccaatcccaccacgaagggacacagtgggtgtggcgtgctgtccagggcacttggttatcctgcagtcccctgggttctctccttctcttccaggacgcagacacagtgcggcaccatgtggtcctgggggaggccctctccatggaaaccctgcggaagggtggacaccgcaactccctcctgggccctgcccactggatcgtcttctacaaccacagtggccaggtttgggggtcagggagcaaggagaccctagcccgggcccctacccctgaagatcccttccctttggagtttccctagctgtgagcctccaacctctaacctctgctccagggagccccccggccctggagtactgcctgaccttacagccctgccaggtgagggtggtaggtgggtggattcagcctccatccctctccccagcctgaggtgaaccatgtgccactggaaggccccatgctggaggcccctggccgctcgctgattggtctgtcgggggtcctgacggtgggctcaagtcgctgcctgcatagccacgctgaggccctgcgggtgagaggctggggccacaggaaggccggcacgggagttcagagctgggagggcctagctgacctgactagtggggaggggcgcatggtcagtctgtcctggaggaagggcagggatccaggcctggactgcctgatgccccaccctttttccctctaggagaaatgtgtaaactgcaccaggagattccgctgcactcagggcttccagctgcaggtgagactgggcttagcgcagctctgtccctgtgttgcctgccctcacttcttccaaggtgcccaatcccctcaccctccagcccagttttgcttggcccaggcatccaggctcagggaactgggtggctgacgtccccatagaggtctggagggctggcctggctctgctgaggggagactctctcagacttgcggcccgaggcttggatcctgtccgccctctcccatcagtctgcctgtcttggtccctctctgacccttctgctgttctgctcagctgcctgacacctgtagtccatctgtctgtctccccattcactgccctgcccctgcccaagccactggctgacccctcctgcctgtccccgtttccaggacacacccaggaagagctgtgtctaccgatctggcttctccttctcccggggctgctcttacacatgtgccaagaagatccaggtttgccctgaagcccccaccccaagcctgtccagagagaaggaggtgggagtgggtgggggcccagggaggagcccagtttggatcttaaggacagagactgggctgggactagggtgaggccagaacccagaggaaggagatgggagagaggtctgaagtcagagggctagtgtttgtccctgtgtccactcccccagggagagggagtggagacggtgagaggaagaaagggcagtccagagctggttccatcagcgaaaccccaggggcagcaacttggcctggcttggcccctcactgtgggatctgggtgcagcccttccttgcttgagcctcagtttccccaccctgccagagggagggttttaggaggtatctctgtgtctctttcagctctgagctgctcatccctgggactggggttctgaggggttttgctccatggctctgtggccccgagatgcccccgttccccttccccctgacaccttgctgggcctctctcgccctctctcccatccccacgccgacaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggaggtgaggactggggaggggcgggggtgggcctcctggcagcagagagcagcctggagaccccagccgggacaggatggaggcactgggcaactcctatcctcctttataccactgcagtgtgtgactgtgcccatgggctgtgccaggaggggctgcaaggggacggaagctgtgtctgtaacgtgggctggcagggcctccgctgtgaccagagtgagtgggtcccaatggggagggggcaggtggctactgaggaggctgttcctgggtctgaatgaccagagtcatcctcctgccccccagaaatcaccagccctcagtgccctaggaagtgcgaccccaatgccaagtgagtgggcccagcctggggcgggtgggtgagtggcggggagcctgggggtcaaggcacgactggacaggcatgggctaagctgctgctaccacccctcccagctgcgtgcaggactcggccggagcctccacctgcgcctgtgctgcgggatactccggcaatggcatcttctgttcaggtccagccacacggatgcccagggccctccctgaaattttggggtggctgtccccacagagcccccttcacttcctctaggcccagcccccctcactgtcccatctcaggactcacccttccctctgaccttttcctttctctcacgccctcctctcctgtcctgcctccggccccgtgcctgctcctgaggactctctggccatctccccttagaggtggacccctgcgcccacggccatgggggctgctcccctcatgccaactgtaccaaggtggcacctgggcagcggacatgcacctgccaggatggctacatgggcgacggggagctgtgccagggtgagactaggcccctaacctgggtttatgttgggctaggggtctggcttcagtgatctgagtcagattaggaagggctgaaggggcacatgggacttgtggggactgggggcgctgagagaggagggggtgggacagatgaaggtacaaaggacagaggaaggggggggccactaagggccacccccatcccagtgtctcttcccccagaaattaacagctgtctcatccaccacgggggctgccacattcacgccgagtgcatccccactggcccccagcaggtcagcatggcagggttggacatggggcatcatgccatgccctcacaggatggggcctcccttcagggcaggcccggggaggggggtgacccttttacccagggtgctggccagctgttggctcccagcagccccactgatcaagactggggacaggtctcctgcagctgccgtgagggttacagcggggatggcatccggacctgcgagctcctggacccctgctctaaggtcaggaccctagtcctgtcctccttcactgacgagaaggagaggggactgaggcaggaaccagcccttctcacctccagcagggtcctaagggcctctgcagggtgggctatcctcccgtcaagctcgagtttaatcagtagcaaataggatctggggggctgagcaggggccttcatggcctctttcacagggctgagtgggatagagaagtagtgaaagccactgataccttcctctcctgtccctgactcagaacaatggaggatgcagcccatatgccacctgcaaaagcacaggggatggccagaggacatgtacctgcgacacagcccacaccgtgggggacggcctcacctgccgtgcccgagtcggcctggtaatgatgcccaagtcagacccctgatctggtcttggctgtgccccatgggcctgacccatggtcttggcaaagatccgatttgatcctgattctggccctgaccatgacactgagcctgacccctgattgtacctgggcctgaccccagctatgacccatgaaactagccctgaccccaactcagaccccgcggctttcctttcctcatgtctgccttggctagaccttgggccgcaggtgctgggacaggcaccaggccctgaatgggggccgccccaaatctgagctgaccctcgcccccccaggagctcctgagggataagcatgcctcattcttcagcctccgcctcctggtgagtggccagcttgggcctctgtgacctgcaagtcccacccctctctggacttctgtccccatctggtcagggggttggctggtttgttctctctgagtccaggtcttccccaggagatccatggacgcctcaggcttctcccattccactcatgctgttgctgcctcccgcaggaatataaggagctcaagggcgatgggcctttcaccatcttcgtgccgcacgcagatctaatgagcaacctgtcgcaggtatgcagcccccagagcgaggctgggcagggctgggtgctctggtggtggccctgcgtgggctgaggcggcagtcagggaccccactcccctcgcgactctgctccaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggaggtgagccctggccctggcctgccccgctccatcccgccccgccccgcccctgcgcgccattgccccaggccccacggcccacgcagtcaagaaccccacctcccctagccagggggcgcctcctgctgagtccagcccgggactccgcccttgacctgggcctcgcccactgctgtgggcctgtcctgcccagcgcccttgtgtccttccgaggactgagccccgccccctgccctgactccgccccagaccccgcccacctcgtcctggtcccgaagaaccgggactgcctgcaccctgacttgcccaacccaggttcaacctccacctcaggccccgatagcttccgagcaccccacctatctgcttcatcagccccgtgccccgcccccagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgagggtatgacaagcacgggcctgggagctggaagacaggcaggtgggggcctgggatcctcccttcccgcctggcctagctgtttatgagagcctttcctcagagaaatgtcaccgccgccgcccagggcttcggttacaagatcttcagcggcctcctgaaggtactgctcccgtgtgggcctgtcattgtggacacacatgcacgtgtaagtgtgtgcaagtgtgtatgcgtacctgtgtgtgcatacccacctgtgtgcacactgtcccgtgtgagcctgtgtgaactcatgtgtgcacaagccacatctgtgtgcatgtgcaccccaggttgagcacatgggtgtgcttatgtgtgcccacattcctgacagcagtgtgcacagttgagatgtgtgcacacacatgcctgcctgggatggtcacagatgagtgtgggtgagtgtggagccccttcctgctctgtggcacagccccctgcctttgctaactagtctctgactgggcagcgactgccacatctttgctgcaccccaccaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggtgggtgcagcccccaaccttggtcttcactgcctgcagctcagcctgcctttccacctccaaccatgactccacttgctcaggccttggcatctgacctgcccaacctgggcccacttcgaaccatgcatgggacccccatctctttctcctgcagccgaacgcgggccgtgagtctggggagagggcttggatgaagggagtaggaggcaggggccctggcagtagcagctggagtgcacctgcgacccttgcatacctcatattctctccttgcagggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactggtgagggaggccagaggcagacgggcagaagcccaccccgcctgtggtgtccatccacctgactttgctgtattggcctccctcccttccagaacacctgcagcatctgtgggctggagccaccctgtcctgaggggtcacaggagcaggtacagggctaggggctgagtgggggtggtggggtgggggcaggcagagcccaaggaccaccaaactcagaaccacccaactgcggcctgactcctttggcccagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaaggtgagcattgcagacactgttgactagctcctctgttccccaagagagccaaggcacagctctggccattgtccttgagagagtgttggggcgtccagggctggggagctctgggtggtgagtagattttactagcgggagtgctcgggagaggcatggcggtagtgtggacggtgcagaacctggggaggagtgctggggggtctggatgttctccaggacatgggcctggagggatcttagcagtgggcggacataggtcagggacagatggcagggctaggctggaggtacgtatatgggggtcctggtgagagcccctggccccacctcacttctcctaccccatccccagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaaggtgggccatccctgcctgccccacggcccgattcctttggcccttgtgggccaatctttatcttcctgcctgtccctctccccagcctgccgctgcactgtgcatggccgctgtgatgagggccttgggggctctggctcctgcttctgtgatgaaggctggactgggccacgctgtgaggtgcaactgggtgagtagccccgtgctcgtgcccaccctacacacttgtgtacatgtgcacacatcagtaaaggcaccaagggtgggtggaacgggcagaggccaggaaggcaactactaacctggttctgggggctgggccctgggggacagagctgcagcctgtgtgtaccccaccctgtgcacccgaggctgtgtgccgtgcaggcaacagctgtgagtgcagcctgggctatgaaggggatggccgtgtgtgtacaggtaagcagatgggcggggacatggaggtgggaggcccccactcccctgcagtcactaggtccaaccactccctccctgccctcagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctggtgagcaggtgggggaaccgagtagccagggtggggaggcctggcagaacttccgacctctgaagggtgaagggcgcccactgctctctccaaccccagaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccagggtgtgtccccctgcccactcccagtacccatttcattcctcagagccggccagctgaccatgcacccctccattctgcagagaaacgggctggcgttttccacctccaggccaccagcggcccttatggtctgaacttttcggaggctgaggcggcatgcgaagcacagggagccgtccttgcttcattccctcagctctctgctgcccagcaggtgtgtggggcccagaagttggggccaagtgttgggggaggccttgactgggtcttgggtcttagtatcccctcctgttcctacagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaaggtgtgtccacccgaccaaaccctacttcccctgctctgccccttcccaccaatctgctgagccactgacctgcttttcctgcagatgtggcctgccgatgccgaaatggcttcgtgggtgacgggatcagcacgtgcaatgggaagctgctggatgtgctggctgccactgccaacttctccaccttctatggggtgtgtgggggccacccttgggggcggggggtgctgggatccccgaggagggagcctgctcatagttctgtctttccaccgtgccagatgctattgggctatgccaatgccacccagcggggtctcgacttcctggacttcctggatgatgagctcacgtataagacactcttcgtccctgtcaatgaaggctttgtggacaacatggtaacccccaagggtgtgggcagagcagagcctgcattggccatctccatctggcccaggccaacaggccttgctctgctcacagacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggtgagtctggccactgtcccaccctgttggccctggccctcgctcacagtgggcctgactctggtctccctgcaggccccagggacagttgtggttagccgtatcattgtgtgggacatcatggccttcaatggcatcatccatgctctggccagccccctcctggcacccccacagcccgtgagttgaggaagggggaggcagagcccttcctggcacccccacaacctgtgagcccggggaaggtggtgggacagctcccgccaggtcaacactctccctgtttgtttgtagcaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggtagggctgggttggggctgccatggcgggtccttggatctcgcttgaggcgcctcgccactcacccctctgctgctcccaggcggaagatgatgctgatgacgacttctcaccgtggcaagaagggaccaaccccaccctggtctctgtccccaaccctgtctttggcagcgacaccttttgtgaacccttcgatgtaagcatggaagtgaagaagtgtggcagatgtgggatgggcccaggccctggtcacaccctccaccaccaaccctgctcttctaggactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcaccaa

SEQ ID NO: 2 - RNA, 7928 nt, Homo sapiens
  source 1..7928: mol_type = mRNA
ctgcctgtgctgtgcctgcctcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggtgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacatgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcgggccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcaccaa

SEQ ID NO: 3 - RNA, 7909 nt, Homo sapiens
  source 1..7909: mol_type = mRNA
gcctcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaaaaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcacc

SEQ ID NO: 4 - RNA, 2474 nt, Homo sapiens
  source 1..2474: mol_type = mRNA
cctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaatgtgagtcccatcctctcctggggtgaggtatggggaacacaaggactccttcaccggttggccagtgactggctgtgtgtctgggccaagcctgccactctggccttgggattctcatatctatgcagtggaaagttggatcaaattatttctcatattttaattaaaaataattagagaggcc

SEQ ID NO: 5 - RNA, 7916 nt, Homo sapiens
  source 1..7916: mol_type = mRNA
agcttgcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgtctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcgggccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcaccaaaaa

SEQ ID NO: 6 - RNA, 7870 nt, Homo sapiens
  source 1..7870: mol_type = mRNA
actctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacacagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggtgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgggaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgcgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgaccagcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcgtagtcagcctgggtgcccgcaagaacctctcagaacgctgggatgcctactgcttccgtgtgcaagatgtggcctgccgatgccgaaatggctttgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccacgggctttggcttctctgccttccaggcggaagatgatgctgacgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaagtgccctcagcggatgtgggc

SEQ ID NO: 7 - RNA, 7874 nt, Homo sapiens
  source 1..7874: mol_type = mRNA
tcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgcgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgaccagcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcgtagtcagcctgggtgcccgcaagaacctctcagaacgctgggatgcctactgcttccgtgtgcaagatgtggcctgccgatgccgaaatggctttgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccacgggctttggcttctctgccttccaggcggaagatgatgctgacgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcacc

SEQ ID NO: 8 - DNA, 7928 bp, Homo sapiens
  source 1..7928: mol_type = other DNA
ctgcctgtgctgtgcctgcctcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggtgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacatgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcgggccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcaccaa

SEQ ID NO: 9 - DNA, 7909 bp, Homo sapiens
  source 1..7909: mol_type = other DNA
gcctcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaaaaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcacc

SEQ ID NO: 10 - DNA, 2474 bp, Homo sapiens
  source 1..2474: mol_type = other DNA
cctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaatgtgagtcccatcctctcctggggtgaggtatggggaacacaaggactccttcaccggttggccagtgactggctgtgtgtctgggccaagcctgccactctggccttgggattctcatatctatgcagtggaaagttggatcaaattatttctcatattttaattaaaaataattagagaggcc

SEQ ID NO: 11 - DNA, 7916 bp, Homo sapiens
  source 1..7916: mol_type = other DNA
agcttgcctagagctcattccctacgccccgactctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgtctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcgggccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgtgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgacctgcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcatagtcagcctgggtgcccgcaagaacctctcagaacgctgggatgcctactgcttccgtgtgcaagatgtggcctgccgatgccgaaatggcttcgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccatgggctttggcttctctgccttccaggcggaagatgatgctgatgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcaccaaaaa

SEQ ID NO: 12 - DNA, 7870 bp, Homo sapiens
  source 1..7870: mol_type = other DNA
actctgtcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacacagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggtgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgggaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgcgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgaccagcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcgtagtcagcctgggtgcccgcaagaacctctcagaacgctgggatgcctactgcttccgtgtgcaagatgtggcctgccgatgccgaaatggctttgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccacgggctttggcttctctgccttccaggcggaagatgatgctgacgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaagtgccctcagcggatgtgggc

SEQ ID NO: 13 - DNA, 7874 bp, Homo sapiens
  source 1..7874: mol_type = other DNA
tcctggacagcgtgcccaccagccatggcggggccccggggcctcctcccactctgcctcctggccttctgcctggcaggcttcagcttcgtcagggggcaggtgctgttcaaaggctgtgatgtgaaaaccacgtttgtcactcatgtaccctgcacctcgtgcgcggccatcaagaagcagacgtgtccctcaggctggctgcgggagctcccggatcagataacccaggactgccgctacgaagtacagctggggggctctatggtgtccatgagcggctgcagacggaagtgccggaagcaagtggtgcagaaggcctgctgccctggctactggggttcccggtgccatgaatgccctgggggcgctgagaccccatgcaatggccacgggacctgcttggatggcatggacaggaatgggacctgtgtgtgccaggaaaacttccgcggctcagcctgccaggagtgccaagaccccaaccggttcgggcctgactgccaatcggtgtgcagctgtgtgcacggagtgtgcaaccatgggccacgtggggatggaagctgcctgtgctttgctggatacactggcccccactgtgatcaagagctgcccgtctgccaggagctgcgctgtccccagaacacccagtgctccgcagaggctcccagctgcaggtgcctgcccggctacacacagcagggcagtgaatgccgagcccccaacccctgctggccatcaccctgctcactgctggcccagtgctcggtgagccccaaggggcaggctcagtgtcactgccctgagaactaccatggcgatgggatggtgtgtctgcccaaggacccatgcactgacaaccttggtggctgccccagcaactctactttgtgtgtgtaccagaagccgggccaggccttctgcacctgccggccaggcctggtcagcatcaacagcaacgcttctgcgggctgcttcgccttctgctcccccttctcctgcgaccggtctgccacttgccaggtgaccgctgatgggaagaccagctgtgtgtgcagggaaagcgaggtgggggatgggcgtgcctgctacggacacctgctccacgaggtgcagaaggccacgcagacaggccgggtgttcctgcagctgagggtcgccgtggccatgatggaccagggctgccgggaaatccttaccacagcgggccctttcaccgtgctggtgccatccgtctcctccttctcctccaggaccatgaatgcatcccttgcccagcagctctgtagacagcacatcatcgcagggcagcacatcctggaggacacaaggacccaacaaacacgaaggtggtggacgctggccgggcaggagatcaccgtcacctttaaccaattcacgaaatactcctacaagtacaaagaccagccccagcagacgttcaacatctacaaggccaacaacatagcagctaatggcgtcttccacgtggtcactggcctgcggtggcaggccccctctgggacccctggggatcccaagagaactatcggacagatcctcgcctctaccgaggccttcagccgctttgaaaccatcctggagaactgtgggctgccctccatcctggacggacctgggcccttcacagtctttgccccaagcaatgaggctgtggacagcttgcgtgacggccgcctgatctacctcttcacagcgggtctctctaaactgcaggagttggtgcggtaccacatctacaaccacggccagctgaccgttgagaagctcatctccaagggtcggatcctcaccatggcgaaccaggtcctggctgtgaacatttctgaggaggggcgcatcctgctgggacccgagggggtcccgctgcagagggtagacgtgatggccgccaatggcgtgatccacatgctggacggcatcctgctgcccccgaccatcctgcccatcctgcccaagcactgcagcgaggagcagcacaagattgtggcgggctcctgtgtggactgccaagccctgaacaccagcacgtgtccccccaacagtgtgaagctggacatcttccccaaggagtgtgtctacatccatgacccaacggggctcaatgtgctaaagaagggctgtgccagctactgcaaccaaaccatcatggaacaaggctgctgcaaaggttttttcgggcctgactgcacgcagtgtcctgggggcttctccaacccctgctatggcaaaggcaattgcagtgatgggatccagggcaatggggcctgcctctgcttcccagactacaagggcatcgcctgccacatctgctcgaacccaaacaagcatggagagcaatgccaggaagactgcggctgtgtccatggtctctgcgacaaccgcccaggcagtgggggggtgtgccagcagggcacgtgtgcccctggcttcagtggccggttctgcaacgagtccatgggggactgtgggcccacagggctggcccagcactgccacctgcatgcccgctgtgttagccaggagggtgttgccagatgtcgctgtcttgatggctttgagggtgatggcttctcctgcacacctagcaacccctgctcccacccggaccgtggaggctgctcagagaatgctgagtgtgtccctgggtccctgggcacccaccactgcacatgccacaaaggctggagtggggatggccgcgtctgtgtggctattgacgagtgtgagctggacgtgagaggtggctgccacaccgatgccctctgcagctatgtgggccccgggcagagccgatgcacctgcaagctgggctttgccggggatggctaccagtgcagccccatcgacccctgccgggcaggcaatggcggctgccacggcctggccacctgccgggcagtggggggaggtcagcgggtctgcacgtgcccccctggctttgggggtgatggcttcagctgttatggagacatcttccgggagctggaggcaaatgcccacttctccatcttctaccaatggcttaagagtgccggcatcacgcttcctgccgaccgccgagtcacagccctggtgccctccgaggctgcagtccgtcagctgagccccgaggaccgagctttctggctgcagccaaggacgctgccgaacctggtcagggcccattttctccagggtgccctcttcgaggaggagctggcccggctgggtgggcaggaagtggccaccctgaaccccaccacacgctgggagattcgcaacattagtgggagggtctgggtgcagaatgccagcgtggatgtggctgacctccttgccaccaacggtgtcctacacatcctcagccaggtcttactgcccccccgaggggatgtgcccggtgggcaggggttgctgcagcagctggacttggtgcctgccttcagcctcttccgggaattgctgcagcaccatgggttggtgccccagattgaggctgccactgcctacaccatctttgtgcccaccaaccgctccctggaggcccagggcaacagcagtcacctggacgcagacacagtgcggcaccatgtggtcctgggggaggccctctccatggaaaccctgcggaagggtggacaccgcaactccctcctgggccctgcccactggatcgtcttctacaaccacagtggccagcctgaggtgaaccatgtgccactggaaggccccatgctggaggcccctggccgctcgctgattggtctgtcgggggtcctgacggtgggctcaagtcgctgcctgcatagccacgctgaggccctgcgggagaaatgtgtaaactgcaccaggagattccgctgcactcagggcttccagctgcaggacacacccaggaagagctgtgtctaccgatctggcttctccttctcccggggctgctcttacacatgtgccaagaagatccaggtgccggactgctgccctggtttctttggcacgctgtgtgagccatgcccagggggtctagggggggtgtgctcaggccatgggcagtgccaggacaggttcctgggcagcggggagtgccactgccacgagggcttccatggaacggcctgtgaggtgtgtgagctgggccgctacgggcccaactgcaccggagtgtgtgactgtgcccatgggctgtgccaggaggggctgcaaggggacggaagctgtgtctgtaacgtgggctggcagggcctccgctgtgaccagaaaatcaccagccctcagtgccctaggaagtgcgaccccaatgccaactgcgtgcaggactcggccggagcctccacctgcgcctgtgctgcgggatactccggcaatggcatcttctgttcagaggtggacccctgcgcccacggccatgggggctgctcccctcatgccaactgtaccaaggtggcacctgggcagcggacatgcacctgccaggatggctacatgggcgacggggagctgtgccaggaaattaacagctgtctcatccaccacgggggctgccacattcacgccgagtgcatccccactggcccccagcaggtctcctgcagctgccgtgagggttacagcggggatggcatccggacctgcgagctcctggacccctgctctaagaacaatggaggatgcagcccatatgccacctgcaaaagcacaggggatggccagaggacatgtacctgcgacacagcccacaccgtgggggacggcctcacctgccgtgcccgagtcggcctggagctcctgagggataagcatgcctcattcttcagcctccgcctcctggaatataaggagctcaagggcgatgggcctttcaccatcttcgtgccgcacgcagatctaatgagcaacctgtcgcaggatgagctggcccggattcgtgcgcatcgccagctggtgtttcgctaccacgtggttggctgtcggcggctgcggagcgaggacctgctggagcaggggtacgccacggccctctcagggcacccactgcgcttcagcgagagggagggcagcatatacctcaatgacttcgcgcgcgtggtgagcagcgaccatgaggccgtgaacggcatcctgcacttcattgaccgtgtcctgctgccccccgaggcgctgcactgggagcctgatgatgctcccatcccgaggagaaatgtcaccgccgccgcccagggcttcggttacaagatcttcagcggcctcctgaaggtggccggcctcctgcccctgcttcgagaggcatcccataggcccttcacaatgctgtggcccacagacgccgcctttcgagctctgcctccggatcgccaggcctggctgtaccatgaggaccaccgtgacaagctagcagccattctgcggggccacatgattcgcaatgtcgaggccttggcatctgacctgcccaacctgggcccacttcgaaccatgcatgggacccccatctctttctcctgcagccgaacgcggcccggtgagctcatggtgggtgaggatgatgctcgcattgtgcagcggcacttgccctttgagggtggcctggcctatggcatcgaccagctgctggagccacctggccttggtgctcgctgtgaccactttgagacccggcccctgcgactgaacacctgcagcatctgtgggctggagccaccctgtcctgaggggtcacaggagcagggcagccctgaggcctgctggcgcttctacccgaagttctggacgtcccctccgctgcactctttgggattacgcagcgtctgggtccaccccagcctttggggtaggccccaaggcctgggcaggggctgccaccgcaattgtgtcaccaccacctggaagcccagctgctgccctggtcactatggcagtgagtgccaagcttgccctggcggccccagcagcccttgtagtgaccgtggcgtgtgcatggacggcatgagtggcagtgggcagtgtctgtgccgttcaggttttgctgggacagcctgtgaactctgtgctcctggtgcctttgggccccattgtcaagcctgccgctgcactgtgcatggccgctgtgatgagggccttgggggctctggctcctgcttctgtgatgaaggctggactgggccacgctgtgaggtgcaactggagctgcagcctgtgtgtaccccaccctgtgcacccgaggctgtgtgccgtgcaggcaacagctgtgagtgcagcctgggctatgaaggggatggccgcgtgtgtacagtggcagacctgtgccaggacgggcatggtggctgcagtgagcacgccaactgtagccaggtaggaacaatggtcacttgtacctgcctgcccgactacgagggtgatggctggagctgccgggcccgcaacccctgcacagatggccaccgcgggggctgcagcgagcacgccaactgcttgagcaccggcctgaacacacggcgctgtgagtgccacgcaggctacgtaggcgatggactgcagtgtctggaggagtcggaaccacctgtggaccgctgcttgggccagccaccgccctgccactcagatgccatgtgcactgaccagcacttccaggagaaacgggctggcgttttccacctccaggccaccagcggcccttatggtctgaacttttcggaggctgaggcggcatgcgaagcacagggagccgtccttgcttcattccctcagctctctgctgcccagcagctgggcttccacctgtgcctcatgggctggctggccaatggctccactgcccaccctgtggttttccctgtggcggactgtggcaatggtcgggtgggcgtagtcagcctgggtgcccgcaagaacctctcagaacgctgggatgcctactgcttccgtgtgcaagatgtggcctgccgatgccgaaatggctttgtgggtgacgggatcagcacgtgcaatgggaagctgctggatgtgctggctgccactgccaacttctccaccttctatgggatgctattgggctatgccaatgccacccagcggggtctcgacttcctggacttcctggatgatgagctcacgtataagacactcttcgtccctgtcaatgaaggctttgtggacaacatgacgctgagtggcccagacttggagctgcatgcctccaacgccaccctcctaagtgccaacgccagccaggggaagttgcttccggcccactcaggcctcagcctcatcatcagtgacgcaggccctgacaacagttcctgggcccctgtggccccagggacagttgtggttagccgtatcattgtgtgggacatcatggccttcaatggcatcatccatgctctggccagccccctcctggcacccccacagccccaggcagtgctggcgcctgaagccccacctgtggcggcaggcgtgggggctgtgcttgccgctggagcactgcttggcttggtggccggagctctctacctccgtgcccgaggcaagcccacgggctttggcttctctgccttccaggcggaagatgatgctgacgacgacttctcaccgtggcaagaagggaccaaccccaccctggtctctgtccccaaccctgtctttggcagcgacaccttttgtgaacccttcgatgactcactgctggaggaggacttccctgacacccagaggatcctcacagtcaagtgacgaggctggggctgaaagcagaagcatgcacagggaggagaccacttttattgcttgtctgggtggatggggcaggaggggctgagggcctgtcccagacaataaaggtgccctcagcggatgtgggccatgtcacc

SEQ ID NO: 14 - protein, 2570 aa, Homo sapiens
MAGPRGLLPLCLLAFCLAGFSFVRGQVLFKGCDVKTTFVTHVPCTSCAAIKKQTCPSGWLRELPDQITQDCRYEVQLGGSMVSMSGCRRKCRKQVVQKACCPGYWGSRCHECPGGAETPCNGHGTCLDGMDRNGTCVCQENFRGSACQECQDPNRFGPDCQSVCSCVHGVCNHGPRGDGSCLCFAGYTGPHCDQELPVCQELRCPQNTQCSAEAPSCRCLPGYTQQGSECRAPNPCWPSPCSLLAQCSVSPKGQAQCHCPENYHGDGMVCLPKDPCTDNLGGCPSNSTLCVYQKPGQAFCTCRPGLVSINSNASAGCFAFCSPFSCDRSATCQVTADGKTSCVCRESEVGDGRACYGHLLHEVQKATQTGRVFLQLRVAVAMMDQGCREILTTAGPFTVLVPSVSSFSSRTMNASLAQQLCRQHIIAGQHILEDTRTQQTRRWWTLAGQEITVTFNQFTKYSYKYKDQPQQTFNIYKANNIAANGVFHVVTGLRWQAPSGTPGDPKRTIGQILASTEAFSRFETILENCGLPSILDGPGPFTVFAPSNEAVDSLRDGRLIYLFTAGLSKLQELVRYHIYNHGQLTVEKLISKGRILTMANQVLAVNISEEGRILLGPEGVPLQRVDVMAANGVIHMLDGILLPPTILPILPKHCSEEQHKIVAGSCVDCQALNTSTCPPNSVKLDIFPKECVYIHDPTGLNVLKKGCASYCNQTIMEQGCCKGFFGPDCTQCPGGFSNPCYGKGNCSDGIQGNGACLCFPDYKGIACHICSNPNKHGEQCQEDCGCVHGLCDNRPGSGGVCQQGTCAPGFSGRFCNESMGDCGPTGLAQHCHLHARCVSQEGVARCRCLDGFEGDGFSCTPSNPCSHPDRGGCSENAECVPGSLGTHHCTCHKGWSGDGRVCVAIDECELDMRGGCHTDALCSYVGPGQSRCTCKLGFAGDGYQCSPIDPCRAGNGGCHGLATCRAVGGGQRVCTCPPGFGGDGFSCYGDIFRELEANAHFSIFYQWLKSAGITLPADRRVTALVPSEAAVRQLSPEDRAFWLQPRTLPNLVRAHFLQGALFEEELARLGGQEVATLNPTTRWEIRNISGRVWVQNASVDVADLLATNGVLHILSQVLLPPRGDVPGGQGLLQQLDLVPAFSLFRELLQHHGLVPQIEAATAYTIFVPTNRSLEAQGNSSHLDADTVRHHVVLGEALSMETLRKGGHRNSLLGPAHWIVFYNHSGQPEVNHVPLEGPMLEAPGRSLIGLSGVLTVGSSRCLHSHAEALREKCVNCTRRFRCTQGFQLQDTPRKSCVYRSGFSFSRGCSYTCAKKIQVPDCCPGFFGTLCEPCPGGLGGVCSGHGQCQDRFLGSGECHCHEGFHGTACEVCELGRYGPNCTGVCDCAHGLCQEGLQGDGSCVCNVGWQGLRCDQKITSPQCPRKCDPNANCVQDSAGASTCACAAGYSGNGIFCSEVDPCAHGHGGCSPHANCTKVAPGQRTCTCQDGYMGDGELCQEINSCLIHHGGCHIHAECIPTGPQQVSCSCREGYSGDGIRTCELLDPCSKNNGGCSPYATCKSTGDGQRTCTCDTAHTVGDGLTCRARVGLELLRDKHASFFSLRLLEYKELKGDGPFTIFVPHADLMSNLSQDELARIRAHRQLVFRYHVVGCRRLRSEDLLEQGYATALSGHPLRFSEREGSIYLNDFARVVSSDHEAVNGILHFIDRVLLPPEALHWEPDDAPIPRRNVTAAAQGFGYKIFSGLLKVAGLLPLLREASHRPFTMLWPTDAAFRALPPDRQAWLYHEDHRDKLAAILRGHMIRNVEALASDLPNLGPLRTMHGTPISFSCSRTRAGELMVGEDDARIVQRHLPFEGGLAYGIDQLLEPPGLGARCDHFETRPLRLNTCSICGLEPPCPEGSQEQGSPEACWRFYPKFWTSPPLHSLGLRSVWVHPSLWGRPQGLGRGCHRNCVTTTWKPSCCPGHYGSECQACPGGPSSPCSDRGVCMDGMSGSGQCLCRSGFAGTACELCAPGAFGPHCQACRCTVHGRCDEGLGGSGSCFCDEGWTGPRCEVQLELQPVCTPPCAPEAVCRAGNSCECSLGYEGDGRVCTVADLCQDGHGGCSEHANCSQVGTMVTCTCLPDYEGDGWSCRARNPCTDGHRGGCSEHANCLSTGLNTRRCECHAGYVGDGLQCLEESEPPVDRCLGQPPPCHSDAMCTDLHFQEKRAGVFHLQATSGPYGLNFSEAEAACEAQGAVLASFPQLSAAQQLGFHLCLMGWLANGSTAHPVVFPVADCGNGRVGIVSLGARKNLSERWDAYCFRVQDVACRCRNGFVGDGISTCNGKLLDVLAATANFSTFYGMLLGYANATQRGLDFLDFLDDELTYKTLFVPVNEGFVDNMTLSGPDLELHASNATLLSANASQGKLLPAHSGLSLIISDAGPDNSSWAPVAPGTVVVSRIIVWDIMAFNGIIHALASPLLAPPQPQAVLAPEAPPVAAGVGAVLAAGALLGLVAGALYLRARGKPMGFGFSAFQAEDDADDDFSPWQEGTNPTLVSVPNPVFGSDTFCEPFDDSLLEEDFPDTQRILTVK

SEQ ID NO: 15 - protein, 803 aa, Homo sapiens
MAGPRGLLPLCLLAFCLAGFSFVRGQVLFKGCDVKTTFVTHVPCTSCAAIKKQTCPSGWLRELPDQITQDCRYEVQLGGSMVSMSGCRRKCRKQVVQKACCPGYWGSRCHECPGGAETPCNGHGTCLDGMDRNGTCVCQENFRGSACQECQDPNRFGPDCQSVCSCVHGVCNHGPRGDGSCLCFAGYTGPHCDQELPVCQELRCPQNTQCSAEAPSCRCLPGYTQQGSECRAPNPCWPSPCSLLAQCSVSPKGQAQCHCPENYHGDGMVCLPKDPCTDNLGGCPSNSTLCVYQKPGQAFCTCRPGLVSINSNASAGCFAFCSPFSCDRSATCQVTADGKTSCVCRESEVGDGRACYGHLLHEVQKATQTGRVFLQLRVAVAMMDQGCREILTTAGPFTVLVPSVSSFSSRTMNASLAQQLCRQHIIAGQHILEDTRTQQTRRWWTLAGQEITVTFNQFTKYSYKYKDQPQQTFNIYKANNIAANGVFHVVTGLRWQAPSGTPGDPKRTIGQILASTEAFSRFETILENCGLPSILDGPGPFTVFAPSNEAVDSLRDGRLIYLFTAGLSKLQELVRYHIYNHGQLTVEKLISKGRILTMANQVLAVNISEEGRILLGPEGVPLQRVDVMAANGVIHMLDGILLPPTILPILPKHCSEEQHKIVAGSCVDCQALNTSTCPPNSVKLDIFPKECVYIHDPTGLNVLKKGCASYCNQTIMEQGCCKGFFGPDCTQCPGGFSNPCYGKGNVSPILSWGEVWGTQGLLHRLASDWLCVWAKPATLALGFSYLCSGKLDQIISHILIKNN

SEQ ID NO: 16 - DNA, 1001 bp, Homo sapiens
  source 1..1001: mol_type = genomic DNA
tctccaccactaaggatgggtgaggacactgctatgtctatcagtattcagaggctggaccttctgtggggtaggcagacactcatggggggcctcccagcacttgacagcccctgggacactttagttcacatctgcataaaaagttcgtgttgttaacaggacattttaaggaacttggcttctttccccaaaaaataaaaaatgtcattggtgtttttgtgttgttttgtttttgttttgagctagagtcttgctctgtggcccagactggagtacagtggtatgatctcagctccctgcaacctctacctcctggggtcaagcgattctcctacctcagcctcccaagtagctgggattacaggtgcctgccaccatgccccgctaagttttgttttttgttgttgttgttgttgttttttgagacagagttttgctctgtcacccaggctggagtgcagtggcgcgatcttggctcactgcaagctctgcctcccaggttcacgccattctcctgcctcagcctcccaagtagctgggactacaggtgcccgccaccacacccagctaattttttgtatttttagtagagacggggtttcaccatgttagccaggatggtctcgatctcctgacctcgtgatccacccgcctcggcctcccaaagtgctgggattacaggcgtgagccaccgcgcccggcctaggttttgtatttttagtagagacagggttttaccacgttggccaggctgatctcgaactcctgacctcaagtgatccaccagcctcagcctcccaaagtgctgggattacaggcatgagccacttcagccggccgtcattggttttttaatgaacaaattgctaagctgtggcaagtggcgggacctttattaattagattgataactaagcttagattgctcctctaagctttagcaatccctggctaagcactcatttgtatctttttccccacggcttgattagaaggtg

SEQ ID NO: 17 - DNA, 1001 bp, Homo sapiens
  source 1..1001: mol_type = genomic DNA
tctccaccactaaggatgggtgaggacactgctatgtctatcagtattcagaggctggaccttctgtggggtaggcagacactcatggggggcctcccagcacttgacagcccctgggacactttagttcacatctgcataaaaagttcgtgttgttaacaggacattttaaggaacttggcttctttccccaaaaaataaaaaatgtcattggtgtttttgtgttgttttgtttttgttttgagctagagtcttgctctgtggcccagactggagtacagtggtatgatctcagctccctgcaacctctacctcctggggtcaagcgattctcctacctcagcctcccaagtagctgggattacaggtgcctgccaccatgccccgctaagttttgttttttgttgttgttgttgttgttttttgagacagagttttgctctgtcacccaggctggagtgcagtggcgcgatcttggctcactgcaagctctgcctcccgggttcacgccattctcctgcctcagcctcccaagtagctgggactacaggtgcccgccaccacacccagctaattttttgtatttttagtagagacggggtttcaccatgttagccaggatggtctcgatctcctgacctcgtgatccacccgcctcggcctcccaaagtgctgggattacaggcgtgagccaccgcgcccggcctaggttttgtatttttagtagagacagggttttaccacgttggccaggctgatctcgaactcctgacctcaagtgatccaccagcctcagcctcccaaagtgctgggattacaggcatgagccacttcagccggccgtcattggttttttaatgaacaaattgctaagctgtggcaagtggcgggacctttattaattagattgataactaagcttagattgctcctctaagctttagcaatccctggctaagcactcatttgtatctttttccccacggcttgattagaaggtg

SEQ ID NO: 18 - DNA, 20 bp, synthetic construct
  misc_feature 1..20: note = Synthetic sequence; gRNA Recognition Sequence
  source 1..20: mol_type = other DNA
gatgtgtaggacaccgttgg

SEQ ID NO: 19 - DNA, 20 bp, synthetic construct
  misc_feature 1..20: note = Synthetic sequence; gRNA Recognition Sequence
  source 1..20: mol_type = other DNA
tctacatccatgacccaacg

SEQ ID NO: 20 - DNA, 20 bp, synthetic construct
  misc_feature 1..20: note = Synthetic sequence; gRNA Recognition Sequence
  source 1..20: mol_type = other DNA
gctggtgtttcgctaccacg

SEQ ID NO: 21 - DNA, 20 bp, synthetic construct
  misc_feature 1..20: note = Synthetic sequence; gRNA Recognition Sequence
  source 1..20: mol_type = other DNA
ggacccaacaaacacgaagg

SEQ ID NO: 22 - DNA, 20 bp, synthetic construct
  misc_feature 1..20: note = Synthetic sequence; gRNA Recognition Sequence
  source 1..20: mol_type = other DNA
gctgtggacagcttgcgtga

SEQ ID NO: 23 - DNA, 20 bp, synthetic construct
  misc_feature 1..20: note = Synthetic sequence; gRNA Recognition Sequence
  source 1..20: mol_type = other DNA
ctgccgtgagggttacagcg

SEQ ID NO: 24 - DNA, 20 bp, synthetic construct
  misc_feature 1..20: note = Synthetic sequence; gRNA Recognition Sequence
  source 1..20: mol_type = other DNA
agctaatggcgtcttccacg

SEQ ID NO: 25 - DNA, 20 bp, synthetic construct
  misc_feature 1..20: note = Synthetic sequence; gRNA Recognition Sequence
  source 1..20: mol_type = other DNA
cggtaccacatctacaacca

SEQ ID NO: 26 - DNA, 20 bp, synthetic construct
  misc_feature 1..20: note = Synthetic sequence; gRNA Recognition Sequence
  source 1..20: mol_type = other DNA
gccgccgacagccaaccacg

SEQ ID NO: 27 - DNA, 20 bp, synthetic construct
  misc_feature 1..20: note = Synthetic sequence; gRNA Recognition Sequence
  source 1..20: mol_type = other DNA
acagatcctcgcctctaccg

SEQ ID NO: 28 - DNA, 20 bp, synthetic construct
  misc_feature 1..20: note = Synthetic sequence; gRNA Recognition Sequence
  source 1..20: mol_type = other DNA
acagcgtgccaaagaaacca

SEQ ID NO: 29 - DNA, 20 bp, synthetic construct
  misc_feature 1..20: note = Synthetic sequence; gRNA Recognition Sequence
  source 1..20: mol_type = other DNA
tagatgttgaacgtctgctg

SEQ ID NO: 30 - DNA, 20 bp, synthetic construct
  misc_feature 1..20: note = Synthetic sequence; gRNA Recognition Sequence
  source 1..20: mol_type = other DNA
caggaatataaggagctcaa

SEQ ID NO: 31 - DNA, 20 bp, synthetic construct
  misc_feature 1..20: note = Synthetic sequence; gRNA Recognition Sequence
  source 1..20: mol_type = other DNA
caaggacccaacaaacacga

SEQ ID NO: 32 - DNA, 20 bp, synthetic construct
  misc_feature 1..20: note = Synthetic sequence; gRNA Recognition Sequence
  source 1..20: mol_type = other DNA
gccagaaagctcggtcctcg

SEQ ID NO: 33 - DNA, 20 bp, synthetic construct
  misc_feature 1..20: note = Synthetic sequence; gRNA Recognition Sequence
  source 1..20: mol_type = other DNA
cgtcaatagccacacagacg

SEQ ID NO: 34 - DNA, 20 bp, synthetic construct
  misc_feature 1..20: note = Synthetic sequence; gRNA Recognition Sequence
  source 1..20: mol_type = other DNA
caccaccaggaaaacttccg

SEQ ID NO: 35 - DNA, 20 bp, synthetic construct
  misc_feature 1..20: note = Synthetic sequence; gRNA Recognition Sequence
  source 1..20: mol_type = other DNA
gaggatgtgtaggacaccgt

SEQ ID NO: 36 - DNA, 20 bp, synthetic construct
  misc_feature 1..20: note = Synthetic sequence; gRNA Recognition Sequence
  source 1..20: mol_type = other DNA
ggatgcactcggcgtgaatg

SEQ ID NO: 37 - DNA, 20 bp, synthetic construct
  misc_feature 1..20: note = Synthetic sequence; gRNA Recognition Sequence
  source 1..20: mol_type = other DNA
gacccatgcactgacaacct

(19) United States
(12) Patent Application Publication
(10) Pub. No.: US 2023/0108957 A1
(43) Pub. Date: Apr. 6, 2023

(54) Treatment Of Psychiatric Disorders And Psychiatric Disorder-Associated MRI Phenotypes With Stabilin 1 (STAB1) Inhibitors

(71) Applicant: Regeneron Pharmaceuticals, Inc., Tarrytown, NY (US)

(72) Inventors: Manuel Allen Revez Ferreira, Tarrytown, NY (US); Veera Rajagopal, Tarrytown, NY (US); Giovanni Coppola, Tarrytown, NY (US); Jonathan Marchini, Tarrytown, NY (US); Aris Baras, Tarrytown, NY (US)

(21) Appl. No.: 17/936,715

(22) Filed: Sep. 29, 2022

Related U.S. Application Data
(60) Provisional application No. 63/251,077, filed on Oct. 1, 2021.

(51) Int. Cl. C12N 15/113; A61P 25/18

(52) U.S. Cl. CPC C12N 15/1138; A61P 25/18

(57) ABSTRACT

The present disclosure provides methods of treating subjects having psychiatric disorders and/or psychiatric disorder-associated MRI phenotypes, and methods of identifying subjects having an increased risk of developing psychiatric disorders and/or psychiatric disorder-associated MRI phenotypes.

### REFERENCE TO SEQUENCE LISTING

This application includes a Sequence Listing submitted electronically as an XML file named 381203590SEQ, created on Sep. 27, 2022, with a size of 156 kilobytes. The Sequence Listing is incorporated herein by reference.

### FIELD

The present disclosure relates generally to the treatment of subjects having psychiatric disorders and/or psychiatric disorder-associated MRI phenotypes with Stabilin 1 (STAB1) inhibitors, and methods of identifying subjects having an increased risk of developing psychiatric disorders and/or psychiatric disorder-associated MRI phenotypes.

### BACKGROUND

Severe affective and behavioral psychiatric disorders affect 5 to 10 million adults in the United States and are the leading cause of disability in North America and Europe. Men and women of all ages and races are at risk for mental illness and for the associated morbidity and societal cost. Although psychopharmacological therapy provides at least partial relief for between 70 to 90% of persons suffering from major depression, bipolar disorder (BPD), obsessive-compulsive disorder (OCD), and panic, and other severe anxiety disorders. Many individuals are not helped or experience unacceptable medication-related side effects. Those experiencing schizophrenia, episodic behavioral disorders, post-traumatic stress disorder (PTSD), addictions, and the behavioral and social disorders associated with autism and pervasive developmental disorders are less often helped by pharmacotherapy or psychotherapy. The economic cost of untreated mental illness is more than 100 billion dollars each year in the United States.

Schizophrenia is a severe and common psychiatric disorder with a lifetime risk of approximately 1% in the general population worldwide. It is a multi-factorial disorder, and its etiology includes strong genetic factors characterized by a complex inheritance pattern. Multiple studies have reported oligodendrocyte and myelin abnormalities, as well as dysregulation of their related genes, in brains of schizophrenia patients.

Bipolar disorder is a chronic disorder that affects 2.3 million adult Americans. Bipolar disorder is characterized by episodes of mania and depression that can last from days to months. Bipolar I disorder is characterized by one or more manic or mixed episodes, usually accompanied by major depressive episodes. Bipolar II disorder is characterized by one or more major depressive episodes accompanied by at least one hypomanic episode. Persons with bipolar disorder usually require lifelong treatment, and recovery between episodes is often poor. Medications are available to treat depression or mania and provide mood stabilization. However, most persons with bipolar disorder require multiple medications to achieve symptom relief. Thus, persons with bipolar disease are at risk for medication-related side effects that prompt some to discontinue therapy. Others who are compliant with therapy do not achieve complete symptom relief.

Cognitive impairment is progressive deterioration of ability to remember, learn new things, concentrate, or make decisions that affect their everyday life. Cognitive impairment ranges from mild to severe. With mild impairment, people may begin to notice changes in cognitive functions, but still be able to do their everyday activities. Severe levels of impairment can lead to losing the ability to understand the meaning or importance of something and the ability to talk or write, resulting in the inability to live independently.

Autism spectrum disorders (ASD) is a range of developmental disorders characterized by trouble with social interaction and communication and by restricted and repetitive behavior. ASD may include impairments regarding joint attention and social reciprocity, challenges with verbal language cues, and poor nonverbal communication skills. Symptoms of ASD generally lead to problems with friendships, romantic relationships, daily living, and vocational success.

The neuroanatomical base for many psychiatric disorders is better understood because of advances in functional neuroimaging, such as Positron Emission Tomography (PET), Magnetic Resonance Imaging (MRI), Functional MRI (fMRI), and Magnetoencephalography (MEG). Recent neuroimaging studies have enriched understanding of the neuroanatomical substrates underlying perception, cognition, and emotion. Data on emotional processes suggest a common neural network involving the prefrontal cortex, amygdala, insula, basal ganglia, and anterior cingulate. MRI is a medical imaging modality in which data are displayed as images that represent planar sections of physical objects such as the human brain. MRI is based on the ability of certain atomic nuclei to absorb and re-emit electromagnetic radiation at certain frequencies, when placed in an external magnetic field.

STAB1 is large, transmembrane receptor protein which may function in angiogenesis, lymphocyte homing, cell adhesion, or receptor scavenging. The protein contains 7 fasciclin, 16 epidermal growth factor (EGF)-like, and 2 laminin-type EGF-like domains as well as a C-type lectin-like hyaluronan-binding Link module. STAB1 is primarily expressed on sinusoidal endothelial cells of liver, spleen, and lymph node. In the brain STAB1 is selectively expressed in microglia cells. STAB1 has been shown to endocytose ligands such as low-density lipoprotein, Gram-positive and Gram-negative bacteria, and advanced glycosylation end products. Supporting its possible role as a scavenger receptor for acetylated low-density lipoprotein, the protein rapidly cycles between the plasma membrane and early endosomes.

### SUMMARY

The present disclosure provides methods of treating subjects having psychiatric disorders and/or psychiatric disorder-associated MRI phenotypes, the methods comprising administering a STAB1 inhibitor to the subjects.

The present disclosure also provides methods of treating subjects having schizophrenia, the methods comprising administering a STAB1 inhibitor to the subjects.

The present disclosure also provides methods of treating subjects having bipolar type II disorder, the methods comprising administering a STAB1 inhibitor to the subjects.

The present disclosure also provides methods of treating subjects having cognitive impairment, the methods comprising administering a STAB1 inhibitor to the subjects.

The present disclosure also provides methods of treating subjects having an autism spectrum disorder, the methods comprising administering a STAB1 inhibitor to the subjects.

The present disclosure also provides methods of treating subjects with a therapeutic agent that treats or inhibits a psychiatric disorder and/or a psychiatric disorder-associated MRI phenotype, the methods comprising the steps of: determining whether the subject has a variant nucleic acid molecule that decreases STAB1 expression and/or activity by: obtaining or having obtained a biological sample from the subject; and performing or having performed a sequence analysis on the biological sample to determine if the subject has a genotype comprising the variant nucleic acid molecule that decreases STAB1 expression and/or activity; and i) administering or continuing to administer the therapeutic agent that treats or inhibits the psychiatric disorder and/or the psychiatric disorder-associated MRI phenotype in a standard dosage amount to a subject that does not have a variant nucleic acid molecule that decreases STAB1 expression and/or activity, and administering a STAB1 inhibitor to the subject; or ii) administering or continuing to administer the therapeutic agent that treats or inhibits the psychiatric disorder and/or the psychiatric disorder-associated MRI phenotype in an amount that is the same as or less than a standard dosage amount to a subject that is heterozygous for the variant nucleic acid molecule that decreases STAB1 expression and/or activity, and administering a STAB1 inhibitor to the subject; wherein the presence of a genotype having the variant nucleic acid molecule that decreases STAB1 expression and/or activity indicates the subject has a decreased risk of developing a psychiatric disorder and/or a psychiatric disorder-associated MRI phenotype.

The present disclosure also provides methods of identifying subjects having an increased risk of developing a psychiatric disorder and/or a psychiatric disorder-associated MRI phenotype, the methods comprising: determining or having determined the presence or absence of a variant nucleic acid molecule that decreases STAB1 expression and/or activity in a biological sample obtained from the subject; wherein the subject has an increased risk of developing the psychiatric disorder and/or the psychiatric disorder-associated MRI phenotype when the subject does not have a variant nucleic acid molecule that decreases STAB1 expression and/or activity, and the subject has a decreased risk of developing the psychiatric disorder and/or the psychiatric disorder-associated MRI phenotype when the subject is heterozygous or homozygous for the variant nucleic acid molecule that decreases STAB1 expression and/or activity.

The present disclosure also provides therapeutic agents that treat or inhibit a psychiatric disorder and/or a psychiatric disorder-associated MRI phenotype for use in the treatment of a psychiatric disorder and/or a psychiatric disorder-associated MRI phenotype (or for use in the preparation of a medicament for treating a psychiatric disorder and/or a psychiatric disorder-associated MRI phenotype) in a subject identified as having a variant nucleic acid molecule that decreases STAB1 expression and/or activity, or the complement thereof, wherein the nucleic acid molecule has a nucleotide sequence comprising a guanine at a position corresponding to position 501 according to SEQ ID NO:17, or the complement thereof.

The present disclosure also provides STAB1 inhibitors for use in the treatment of a psychiatric disorder and/or a psychiatric disorder-associated MRI phenotype (or for use in the preparation of a medicament for treating a psychiatric disorder and/or a psychiatric disorder-associated MRI phenotype) in a subject that: a) does not have a variant nucleic acid molecule that decreases STAB1 expression and/or activity; or b) is heterozygous for a variant genomic nucleic acid molecule that decreases STAB1 expression and/or activity, or the complement thereof, wherein the genomic nucleic acid molecule has a nucleotide sequence comprising a guanine at a position corresponding to position 501 according to SEQ ID NO:17, or the complement thereof.

### BRIEF DESCRIPTION OF THE DRAWINGS

The accompanying FIGURES, which are incorporated in and constitute a part of this specification, illustrate several features of the present disclosure.

FIG. 1 shows STAB1 gene burden associations with depression and other psychiatric traits.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A common variant located near the STAB1 gene associated with a decreased risk of developing a psychiatric disorder and/or a psychiatric disorder-associated MRI phenotype in humans has been identified in accordance with the present disclosure. For example, the rs11921116 single nucleotide polymorphism, a genetic alteration that changes the adenine at position 501 in the genomic locus (see, SEQ ID NO:16) to a guanine, has been observed to indicate that the subject having such an alteration may have a decreased risk of developing a psychiatric disorder and/or a psychiatric disorder-associated MRI phenotype, such as schizophrenia, bipolar type II disorder, cognitive impairment, or an autism spectrum disorder. It is believed that no variants at or near the STAB1 gene or protein have any known association with the psychiatric disorders and/or psychiatric disorder-associated MRI phenotypes cognitive impairment or an autism spectrum disorder. Altogether, the genetic analyses described herein surprisingly indicate that the STAB1 gene and, in particular, a variant near the STAB1 gene, associates with a decreased risk of developing psychiatric disorders and/or psychiatric disorder-associated MRI phenotypes, such as schizophrenia, bipolar type II disorder, cognitive impairment, or an autism spectrum disorder. Moreover, the identification by the present disclosure of the association between additional variants and gene burden masks indicates that decreased STAB1 expression and/or activity is responsible for a protective effect in psychiatric disorders and/or psychiatric disorder-associated MRI phenotypes. Therefore, subjects that lack a variant nucleic acid molecule that decreases STAB1 expression and/or activity that have an increased risk of developing psychiatric disorders and/or psychiatric disorder-associated MRI phenotypes, such as schizophrenia, bipolar type II disorder, cognitive impairment, or an autism spectrum disorder, may be treated such that the psychiatric disorders and/or psychiatric disorder-associated MRI phenotypes is prevented, the symptoms thereof are reduced, and/or development of symptoms is repressed. Accordingly, the present disclosure provides methods of leveraging the identification of such variants in subjects to identify or stratify risk in such subjects of developing psychiatric disorders and/or psychiatric disorder-associated MRI phenotypes, such as schizophrenia, bipolar type II disorder, cognitive impairment, or an autism spectrum disorder, or to diagnose subjects as having an increased risk of developing psychiatric disorders and/or psychiatric disorder-associated MRI phenotypes, such as schizophrenia, bipolar type II disorder, cognitive impairment, or an autism spectrum disorder, such that subjects at risk or subjects with active disease may be treated accordingly.

For purposes of the present disclosure, any particular subject can be categorized as having one of three genotypes: i) the subject does not have a variant nucleic acid molecule that decreases STAB1 expression and/or activity; ii) the subject is heterozygous for a variant nucleic acid molecule that decreases STAB1 expression and/or activity; or iii) the subject is homozygous for a variant nucleic acid molecule that decreases STAB1 expression and/or activity. A subject is considered to be reference when the subject does not have a copy of a variant nucleic acid molecule that decreases STAB1 expression and/or activity. A subject is heterozygous for a variant nucleic acid molecule when the subject has a single copy of a variant nucleic acid molecule that decreases STAB1 expression and/or activity. As used herein, a STAB1 variant nucleic acid molecule is any nucleic acid molecule (such as, a genomic nucleic acid molecule, an mRNA molecule, or a cDNA molecule) that contains one or more variations from a wild type nucleic acid molecule, wherein these variations result in impaired expression and/or activity of STAB1. In some embodiments, a variant nucleic acid molecule is any nucleic acid molecule (such as, a genomic nucleic acid molecule, an mRNA molecule, or a cDNA molecule) encoding a STAB1 polypeptide having a partial loss-of-function, a complete loss-of-function, a predicted partial loss-of-function, or a predicted complete loss-of-function. A subject who has a variant nucleic acid molecule that has reduced STAB1 expression level or encodes a STAB1 predicted loss-of-function polypeptide having a partial loss-of-function (or predicted partial loss-of-function) is hypomorphic for STAB1. A subject is homozygous for a variant nucleic acid molecule when the subject has two copies of a variant nucleic acid molecule that decreases STAB1 expression and/or activity.

For subjects that are genotyped or determined not to have a variant nucleic acid molecule that decreases STAB1 expression and/or activity, such subjects have an increased risk of developing psychiatric disorders and/or psychiatric disorder-associated MRI phenotypes, such as schizophrenia, bipolar type II disorder, cognitive impairment, or an autism spectrum disorder. For subjects that are genotyped or determined to be: i) not to have a variant nucleic acid molecule that decreases STAB1 expression and/or activity, or ii) heterozygous for a variant nucleic acid molecule that decreases STAB1 expression and/or activity, such subjects can be treated with a STAB1 inhibitor.

In any of the embodiments described throughout the present disclosure, the variant nucleic acid molecule can be any nucleic acid molecule that decreases STAB1 expression and/or activity (such as, for example, genomic nucleic acid molecule, mRNA molecule, or cDNA molecule). Examples include those encoding a STAB1 polypeptide having a partial loss-of-function, a complete loss-of-function, a predicted partial loss-of-function, or a predicted complete loss-of-function, or a decreased activity of STAB1 polypeptide.

In any of the embodiments described throughout the present disclosure, the STAB1 predicted loss-of-function polypeptide can be any STAB1 polypeptide having a partial loss-of-function, a complete loss-of-function, a predicted partial loss-of-function, or a predicted complete loss-of-function. In any of the embodiments described throughout the present disclosure, the STAB1 predicted loss-of-function polypeptide can be any of the STAB1 polypeptides described herein.

In any of the embodiments described throughout the present disclosure, the psychiatric disorder and/or the psychiatric disorder-associated MRI phenotype can be schizophrenia, bipolar type II disorder, cognitive impairment, or an autism spectrum disorder. In any of the embodiments described throughout the present disclosure, the psychiatric disorder is schizophrenia. In any of the embodiments described throughout the present disclosure, the psychiatric disorder is bipolar type II disorder. In any of the embodiments described throughout the present disclosure, the psychiatric disorder is cognitive impairment. In any of the embodiments described throughout the present disclosure, the psychiatric disorder is an autism spectrum disorder.

Symptoms of schizophrenia include, but are not limited to, delusions, hallucinations, disorganized speech, and disorganized behavior.

Symptoms of bipolar type II disorder include, but are not limited to, depressive symptoms including sadness or hopelessness and hypomanic symptoms including a persistently elevated or irritable mood.

Symptoms of cognitive impairment include, but are not limited to, memory loss, language problems, impaired attention, impaired reasoning and judgement, and impaired complex decision making.

Symptoms of autism spectrum disorder include, but are not limited to, making little or inconsistent eye contact, tending not to look at or listen to people, rarely sharing enjoyment of objects or activities by pointing or showing things to others, failing to, or being slow to, respond to someone calling their name or to other verbal attempts to gain attention, having difficulties with the back and forth of conversation, having facial expressions, movements, and gestures that do not match what is being said having an unusual tone of voice, having trouble understanding another person's point of view or being unable to predict or understand other people's actions.

Psychiatric disorder-associated MRI phenotypes include, but are not limited to, a decrease in volume and/or shape of the left putamen, the right putamen, or both, the right caudate, the left caudate, or both.

In any of the embodiments described herein, the treatment methods described herein can be carried out to alter psychiatric disorder-associated MRI phenotypes in subjects. For example, the treatment methods described herein can be used to increase the volume of grey matter in the brain of a subject, decrease the rate of loss of grey matter in a brain, or decrease the rate and/or amount of age-associated grey matter volume loss in the brain of a subject. The grey matter can be present in any region of the brain, and particularly in the basal ganglia (putamen, caudate regions, etc.). As determined herein, the loss of function of STAB1 increased grey matter volume and may decelerate the age-associated grey matter volume loss. In some embodiments, the psychiatric disorder-associated MRI phenotype is a bipolar disorder, such as bipolar type II disorder. In addition, median T2star and mean intensity MRI phenotypes may be used to confirm an association in basal ganglia changes and STAB1 rare variants.

The present disclosure provides methods of treating a subject having a psychiatric disorder and/or a psychiatric disorder-associated MRI phenotype, the methods comprising administering a STAB1 inhibitor to the subject.

The present disclosure also provides methods of treating a subject having schizophrenia, the methods comprising administering a STAB1 inhibitor to the subject.

The present disclosure also provides methods of treating a subject having bipolar type II disorder, the methods comprising administering a STAB1 inhibitor to the subject.

The present disclosure also provides methods of treating a subject having cognitive impairment, the methods comprising administering a STAB1 inhibitor to the subject.

The present disclosure also provides methods of treating a subject having an autism spectrum disorder, the methods comprising administering a STAB1 inhibitor to the subject.

In some embodiments, the STAB1 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of a STAB1 nucleic acid molecule, such as an mRNA molecule. In some embodiments, the inhibitory nucleic acid molecule hybridizes to a sequence within a STAB1 genomic nucleic acid molecule or mRNA molecule and decreases expression and/or activity of the STAB1 polypeptide in a cell in the subject. In some embodiments, the STAB1 inhibitor comprises an antisense molecule that hybridizes to a STAB1 genomic nucleic acid molecule or mRNA molecule and decreases expression and/or activity of the STAB1 polypeptide in a cell in the subject. In some embodiments, the STAB1 inhibitor comprises an siRNA that hybridizes to a STAB1 genomic nucleic acid molecule or mRNA molecule and decreases expression and/or activity of the STAB1 polypeptide in a cell in the subject. In some embodiments, the STAB1 inhibitor comprises an shRNA that hybridizes to a STAB1 genomic nucleic acid molecule or mRNA molecule and decreases expression and/or activity of the STAB1 polypeptide in a cell in the subject.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (l), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O [(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

In some embodiments, the STAB1 inhibitor comprises a nuclease agent that induces one or more nicks or double-strand breaks at a recognition sequence(s) or a DNA-binding protein that binds to a recognition sequence within a STAB1 genomic nucleic acid molecule. The recognition sequence can be located within a coding region of the STAB1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the STAB1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a STAB1 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of STAB1 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a STAB1 genomic nucleic acid molecule or it can be a nickase that creates a single-strand break in a STAB1 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of a STAB1 genomic nucleic acid molecules can be generated by contacting a cell with a Cas protein and one or more gRNAs that hybridize to one or more gRNA recognition sequences within a target genomic locus in the STAB1 genomic nucleic acid molecule. For example, a gRNA recognition sequence can be located within a region of SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of the genomic locus comprising SEQ ID NO:1. The gRNA recognition sequence can include or be proximate to the start codon of a STAB1 genomic nucleic acid molecule or the stop codon of a STAB1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a STAB1 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a STAB1 genomic nucleic acid molecule. An exemplary gRNA is a gRNA effective to direct a Cas enzyme to bind to or cleave a STAB1 genomic nucleic acid molecule, wherein the gRNA comprises a DNA-targeting segment that hybridizes to a gRNA recognition sequence within the STAB1 genomic nucleic acid molecule that includes or is proximate to the genomic locus comprising SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from the genomic locus comprising SEQ ID NO:1. Other exemplary gRNAs comprise a DNA-targeting segment that hybridizes to a gRNA recognition sequence present within a STAB1 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the STAB1 reference gene are set forth in Table 1 as SEQ ID NOs:18-37.

TABLE 1
Guide RNA Recognition Sequences Near STAB1

| | | SEQ |
|---|---|---|
| | gRNA Recognition | ID |
| Strand | Sequence | NO: |
| − | GATGTGTAGGACACCGTTGG | 18 |
| + | TCTACATCCATGACCCAACG | 19 |
| + | GCTGGTGTTTCGCTACCACG | 20 |
| + | GGACCCAACAAACACGAAGG | 21 |
| + | GCTGTGGACAGCTTGCGTGA | 22 |
| + | CTGCCGTGAGGGTTACAGCG | 23 |
| + | AGCTAATGGCGTCTTCCACG | 24 |
| + | CGGTACCACATCTACAACCA | 25 |
| − | GCCGCCGACAGCCAACCACG | 26 |
| + | ACAGATCCTCGCCTCTACCG | 27 |
| − | ACAGCGTGCCAAAGAAACCA | 28 |
| − | TAGATGTTGAACGTCTGCTG | 29 |
| + | CAGGAATATAAGGAGCTCAA | 30 |
| + | CAAGGACCCAACAAACACGA | 31 |
| − | GCCAGAAAGCTCGGTCCTCG | 32 |
| − | CGTCAATAGCCACACAGACG | 33 |
| + | CACCACCAGGAAAACTTCCG | 34 |
| − | GAGGATGTGTAGGACACCGT | 35 |
| − | GGATGCACTCGGCGTGAATG | 36 |
| + | GACCCATGCACTGACAACCT | 37 |

The Cas protein and the gRNA form a complex, and the Cas protein cleaves the target STAB1 genomic nucleic acid molecule. The Cas protein can cleave the nucleic acid molecule at a site within or outside of the nucleic acid sequence present in the target STAB1 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the STAB1 genomic nucleic acid molecule to which a DNA-targeting segment of a gRNA will bind.

Such methods can result, for example, in a STAB1 genomic nucleic acid molecule in which a region of SEQ ID NO:16 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the STAB1 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the STAB1 inhibitor comprises a small molecule. In some embodiments, the STAB1 inhibitor is an inhibitory nucleic acid molecule. In some embodiments, the STAB1 inhibitor is an antibody, such as a monoclonal antibody.

In some embodiments, the methods of treatment further comprise detecting the presence or absence of a variant nucleic acid molecule that decreases STAB1 expression and/or activity in a biological sample obtained from the subject. As used throughout the present disclosure, “a variant nucleic acid molecule” is any nucleic acid molecule (such as, for example, genomic nucleic acid molecule, mRNA molecule, or cDNA molecule) that contains one or more variations from a wild type nucleic acid molecule, wherein these variations result in impaired expression and/or activity of STAB1. In some embodiments, a variant nucleic acid molecule is any nucleic acid molecule (such as, a genomic nucleic acid molecule, an mRNA molecule, or a cDNA molecule) encoding or resulting in a STAB1 polypeptide having a partial loss-of-function, a complete loss-of-function, a predicted partial loss-of-function, or a predicted complete loss-of-function, or decreased expression thereof.

The present disclosure also provides methods of treating a subject with a therapeutic agent that treats or inhibits a psychiatric disorder and/or a psychiatric disorder-associated MRI phenotype. In some embodiments, the subject has a psychiatric disorder and/or a psychiatric disorder-associated MRI phenotype. In some embodiments, the methods comprise determining whether the subject has a variant nucleic acid molecule that decreases STAB1 expression and/or activity by obtaining or having obtained a biological sample from the subject, and performing or having performed a sequence analysis on the biological sample to determine if the subject has a genotype comprising the variant nucleic acid molecule that decreases STAB1 expression and/or activity. When the subject does not have a variant nucleic acid molecule that decreases STAB1 expression and/or activity, the therapeutic agent that treats or inhibits the psychiatric disorder and/or the psychiatric disorder-associated MRI phenotype is administered or continued to be administered to the subject in a standard dosage amount, and a STAB1 inhibitor is administered to the subject. When the subject is heterozygous for a variant nucleic acid molecule that decreases STAB1 expression and/or activity, the therapeutic agent that treats or inhibits the psychiatric disorders and/or the psychiatric disorder-associated MRI phenotype is administered or continued to be administered to the subject in an amount that is the same as or less than a standard dosage amount, and a STAB1 inhibitor is administered to the subject. The presence of a genotype having a variant nucleic acid molecule that decreases STAB1 expression and/or activity indicates the subject has a decreased risk of developing a psychiatric disorder and/or a psychiatric disorder-associated MRI phenotype. In some embodiments, the subject does not have a variant nucleic acid molecule that decreases STAB1 expression and/or activity. In some embodiments, the subject is heterozygous for the variant nucleic acid molecule that decreases STAB1 expression and/or activity.

For subjects that are genotyped or determined to: i) not have a variant nucleic acid molecule that decreases STAB1 expression and/or activity, or ii) be heterozygous for a variant nucleic acid molecule that decreases STAB1 expression and/or activity, such subjects can be treated with a STAB1 inhibitor, as described herein.

Detecting the presence or absence of a variant nucleic acid molecule that decreases STAB1 expression and/or activity in a biological sample from a subject and/or determining whether a subject has a variant nucleic acid molecule that decreases STAB1 expression and/or activity can be carried out by any of the methods described herein. In some embodiments, these methods can be carried out in vitro. In some embodiments, these methods can be carried out in situ. In some embodiments, these methods can be carried out in vivo. In any of these embodiments, the variant nucleic acid molecule can be present within a cell obtained from the subject.

In some embodiments, when the subject does not have a variant nucleic acid molecule that decreases STAB1 expression and/or activity, the subject is also administered a therapeutic agent that treats or inhibits a psychiatric disorder and/or a psychiatric disorder-associated MRI phenotype in a standard dosage amount. In some embodiments, when the subject is heterozygous for a variant nucleic molecule that decreases STAB1 expression and/or activity, the subject is also administered a therapeutic agent that treats or inhibits a psychiatric disorder and/or a psychiatric disorder-associated MRI phenotype in a dosage amount that is the same as or less than a standard dosage amount.

In some embodiments, the treatment methods further comprise detecting the presence or absence (or level) of a STAB1 polypeptide in a biological sample from the subject. In some embodiments, when the subject does not have a decreased expression and/or activity of a STAB1 polypeptide, the subject is also administered a therapeutic agent that treats or inhibits a psychiatric disorder and/or a psychiatric disorder-associated MRI phenotype in a standard dosage amount. In some embodiments, when the subject has a decreased expression and/or activity of a STAB1 polypeptide, the subject is also administered a therapeutic agent that treats or inhibits a psychiatric disorder and/or a psychiatric disorder-associated MRI phenotype in a dosage amount that is the same as or less than a standard dosage amount.

The present disclosure also provides methods of treating a subject with a therapeutic agent that treats or inhibits a psychiatric disorder and/or a psychiatric disorder-associated MRI phenotype. In some embodiments, the subject has a psychiatric disorder and/or a psychiatric disorder-associated MRI phenotype. In some embodiments, the method comprises determining whether the subject has a decreased expression and/or activity of a STAB1 polypeptide by obtaining or having obtained a biological sample from the subject, and performing or having performed an assay on the biological sample to determine if the subject has a decreased expression and/or activity of a STAB1 polypeptide. When the subject does not have a decreased expression and/or activity of a STAB1 polypeptide, the therapeutic agent that treats or inhibits the psychiatric disorder and/or the psychiatric disorder-associated MRI phenotype is administered or continued to be administered to the subject in a standard dosage amount, and a STAB1 inhibitor is administered to the subject. When the subject has a decreased expression and/or activity of a STAB1 polypeptide, the therapeutic agent that treats or inhibits the psychiatric disorder and/or the psychiatric disorder-associated MRI phenotype is administered or continued to be administered to the subject in an amount that is the same as or less than a standard dosage amount, and a STAB1 inhibitor is administered to the subject. The presence of a decreased expression and/or activity of a STAB1 polypeptide indicates the subject has a decreased risk of developing a psychiatric disorder and/or a psychiatric disorder-associated MRI phenotype. In some embodiments, the subject has a decreased expression and/or activity of a STAB1 polypeptide. In some embodiments, the subject does not have a decreased expression and/or activity of a STAB1 polypeptide.

Detecting the presence or absence (or level) of a STAB1 polypeptide in a biological sample from a subject and/or determining whether a subject has a decreased expression (or function) of a STAB1 polypeptide can be carried out by any of the methods described herein. In some embodiments, these methods can be carried out in vitro. In some embodiments, these methods can be carried out in situ. In some embodiments, these methods can be carried out in vivo. In any of these embodiments, the STAB1 polypeptide can be present within a cell obtained from the subject.

Examples of therapeutic agents that treat or inhibit schizophrenia include, but are not limited to: first generation antipsychotics (chlorpromazine, fluphenazine, haloperidol, perphenazine, thioridazine, thiothixene, trifluoperazine, and loxapine); second generation antipsychotics (asenapine, cariprazine, clozapine, iloperidone, lurasidone, olanzapine, olanzapine/samidorphan, paliperidone, paliperidone palmitate, quetiapine, risperidone, and ziprasidone); and serotonin-dopamine activity modulators (brexpiprazole, aripiprazole, and lumateperone), or any combination thereof.

Examples of therapeutic agents that treat or inhibit bipolar type II disorder include, but are not limited to: mood stabilizers (such as lithium, carbamazepine, lamotrigine, or valproate); antipsychotics (such as aripiprazole, asenapine, cariprazine, quetiapine, olanzapine, risperidone, or ziprasidone); benzodiazepines (such as alprazolam, diazepam, or lorazepam); and antidepressants (such as fluoxetine, paroxetine, or sertraline), or any combination thereof.

Examples of therapeutic agents that treat or inhibit cognitive impairment include, but are not limited to: amitriptyline, benzodiazepines (such as alprazolam, diazepam, or lorazepam), topiramate, paracetamol, levetiracetam, quetiapine, topiramate carbamazepine, fluoxetine, gabapentin, lamotrigine, mirtazapine, olanzapine, opioids, risperidone, sertraline, trazodone, and valproic acid, or any combination thereof.

Examples of therapeutic agents that treat or inhibit autism spectrum disorders include, but are not limited to: second generation antipsychotics (risperidone, aripiprazole, and clozapine), haloperidol, sertraline, oxytocin, secretin, methylphenidate, venlafaxine, selective serotonin reuptake inhibitors (fluoxetine, citalopram, and escitalopram), bumetanide, memantine, rivastigmine, mirtazapine, and melatonin, or any combination thereof.

In some embodiments, the dose of the therapeutic agents that treat or inhibit psychiatric disorders and/or psychiatric disorder-associated MRI phenotypes can be reduced by about 10%, by about 20%, by about 30%, by about 40%, by about 50%, by about 60%, by about 70%, by about 80%, or by about 90% for subjects that are heterozygous for a variant nucleic acid molecule that decreases STAB1 expression and/or activity (i.e., a less than the standard dosage amount) compared to subjects that do not have a variant nucleic acid molecule that decreases STAB1 expression and/or activity (who may receive a standard dosage amount). In some embodiments, the dose of the therapeutic agents that treat or inhibit psychiatric disorders and/or psychiatric disorder-associated MRI phenotypes can be reduced by about 10%, by about 20%, by about 30%, by about 40%, or by about 50%. In addition, the dose of therapeutic agents that treat or inhibit psychiatric disorders and/or psychiatric disorder-associated MRI phenotypes in subjects that are heterozygous for a variant nucleic acid molecule that decreases STAB1 expression and/or activity can be administered less frequently compared to subjects that do not have a variant nucleic acid molecule that decreases STAB1 expression and/or activity.

Administration of the therapeutic agents that treat or inhibit psychiatric disorders and/or psychiatric disorder-associated MRI phenotypes and/or STAB1 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or inhibit psychiatric disorders and/or psychiatric disorder-associated MRI phenotypes and/or STAB1 inhibitors can be administered sequentially or at the same time. In addition, the therapeutic agents that treat or inhibit psychiatric disorders and/or psychiatric disorder-associated MRI phenotypes and/or STAB1 inhibitors can be administered in separate compositions or can be administered together in the same composition.

Administration of the therapeutic agents that treat or inhibit psychiatric disorders and/or psychiatric disorder-associated MRI phenotypes and/or STAB1 inhibitors can occur by any suitable route including, but not limited to, parenteral, intravenous, oral, subcutaneous, intra-arterial, intracranial, intrathecal, intraperitoneal, topical, intranasal, or intramuscular. In some embodiments, the administration is via intrathecal injection. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in psychiatric disorders and/or psychiatric disorder-associated MRI phenotypes, a decrease/reduction in the severity of psychiatric disorders and/or psychiatric disorder-associated MRI phenotypes (such as, for example, a reduction or inhibition of development of psychiatric disorders and/or psychiatric disorder-associated MRI phenotypes), a decrease/reduction in symptoms and psychiatric disorders and/or psychiatric disorder-associated MRI phenotypes-related effects, delaying the onset of symptoms and psychiatric disorders and/or psychiatric disorder-associated MRI phenotypes-related effects, reducing the severity of symptoms of psychiatric disorders and/or psychiatric disorder-associated MRI phenotypes-related effects, reducing the severity of an acute episode, reducing the number of symptoms and psychiatric disorders and/or psychiatric disorder-associated MRI phenotypes-related effects, reducing the latency of symptoms and psychiatric disorders and/or psychiatric disorder-associated MRI phenotypes-related effects, an amelioration of symptoms and psychiatric disorders and/or psychiatric disorder-associated MRI phenotypes-related effects, reducing secondary symptoms, reducing secondary infections, preventing relapse to psychiatric disorders and/or psychiatric disorder-associated MRI phenotypes, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of psychiatric disorders and/or psychiatric disorder-associated MRI phenotypes development/progression (such as, for example, a complete or partial avoidance/inhibition or a delay), and an increased survival time of the affected host animal, following administration of a therapeutic protocol. Treatment of psychiatric disorders and/or psychiatric disorder-associated MRI phenotypes encompasses the treatment of subjects already diagnosed as having any form of psychiatric disorders and/or psychiatric disorder-associated MRI phenotypes at any clinical stage or manifestation, the delay of the onset or evolution or aggravation or deterioration of the symptoms or signs of psychiatric disorders and/or psychiatric disorder-associated MRI phenotypes, and/or preventing and/or reducing the severity of psychiatric disorders and/or psychiatric disorder-associated MRI phenotypes.

The present disclosure also provides methods of identifying a subject having an increased risk of developing a psychiatric disorder and/or a psychiatric disorder-associated MRI phenotype. In some embodiments, the methods comprise determining or having determined the presence or absence of a variant nucleic acid molecule (such as a genomic nucleic acid molecule, mRNA molecule, and/or cDNA molecule) that decreases STAB1 expression and/or activity, in a biological sample obtained from the subject. When the subject lacks a variant nucleic acid molecule that decreases STAB1 expression and/or activity, then the subject has an increased risk of developing a psychiatric disorder and/or a psychiatric disorder-associated MRI phenotype. When the subject has a variant nucleic acid molecule that decreases STAB1 expression and/or activity, then the subject has a decreased risk of developing a psychiatric disorder and/or a psychiatric disorder-associated MRI phenotype compared to a subject that does not have a variant nucleic acid molecule that decreases STAB1 expression and/or activity.

Having a single copy of a variant nucleic acid molecule that decreases STAB1 expression and/or activity is more protective of a subject from developing a psychiatric disorder and/or a psychiatric disorder-associated MRI phenotype than having no copies of a variant nucleic acid molecule that decreases STAB1 expression and/or activity. Without intending to be limited to any particular theory or mechanism of action, it is believed that a single copy of a variant nucleic acid molecule that decreases STAB1 expression and/or activity is protective of a subject from developing a psychiatric disorder and/or a psychiatric disorder-associated MRI phenotype, and it is also believed that having two copies of a variant nucleic acid molecule that decreases STAB1 expression and/or activity may be more protective of a subject from developing a psychiatric disorder and/or a psychiatric disorder-associated MRI phenotype, relative to a subject with a single copy. Thus, in some embodiments, a single copy of a variant nucleic acid molecule that decreases STAB1 expression and/or activity may not be completely protective, but instead, may be partially or incompletely protective of a subject from developing a psychiatric disorder and/or a psychiatric disorder-associated MRI phenotype. While not desiring to be bound by any particular theory, there may be additional factors or molecules involved in the development of psychiatric disorders and/or psychiatric disorder-associated MRI phenotypes that are still present in a subject having a single copy of a variant nucleic acid molecule that decreases STAB1 expression and/or activity, thus resulting in less than complete protection from the development of a psychiatric disorder and/or a psychiatric disorder-associated MRI phenotype.

Detecting the presence or absence of a variant nucleic acid molecule that decreases STAB1 expression and/or activity in a biological sample from the subject and/or determining whether a subject has a variant nucleic acid molecule that decreases STAB1 expression and/or activity can be carried out by any of the methods described herein. In some embodiments, these methods can be carried out in vitro. In some embodiments, these methods can be carried out in situ. In some embodiments, these methods can be carried out in vivo. In any of these embodiments, the variant nucleic acid molecule can be present within a cell obtained from the subject.

In some embodiments, when a subject is identified as having an increased risk of developing a psychiatric disorder and/or a psychiatric disorder-associated MRI phenotype, the subject is further treated with a therapeutic agent that treats or inhibits a psychiatric disorder and/or a psychiatric disorder-associated MRI phenotype and/or a STAB1 inhibitor, as described herein. For example, when the subject does not have a variant nucleic acid molecule that decreases STAB1 expression and/or activity, and therefore has an increased risk of developing a psychiatric disorder and/or a psychiatric disorder-associated MRI phenotype, the subject is administered a STAB1 inhibitor. In some embodiments, such a subject is also administered a therapeutic agent that treats or inhibits a psychiatric disorder and/or a psychiatric disorder-associated MRI phenotype. In some embodiments, when the subject is heterozygous for a variant nucleic acid molecule that decreases STAB1 expression and/or activity, the subject is administered the therapeutic agent that treats or inhibits a psychiatric disorder and/or a psychiatric disorder-associated MRI phenotype in a dosage amount that is the same as or less than a standard dosage amount, and is also administered a STAB1 inhibitor. In some embodiments, the subject does not have a variant nucleic acid molecule that decreases STAB1 expression and/or activity. In some embodiments, the subject is heterozygous for a variant nucleic acid molecule that decreases STAB1 expression and/or activity.

The present disclosure also provides methods of detecting the presence or absence of a variant nucleic acid molecule that decreases STAB1 expression and/or activity in a biological sample obtained from a subject. It is understood that gene sequences within a population and mRNA molecules encoded by such genes can vary due to polymorphisms such as single-nucleotide polymorphisms. The sequences provided herein for the STAB1 variant genomic nucleic acid molecule, STAB1 variant mRNA molecule, and STAB1 variant cDNA molecule are only exemplary sequences. Other sequences for the STAB1 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. In some embodiments, the biological sample comprises blood. The biological sample used in the methods disclosed herein can vary based on the assay format, nature of the detection method, and the tissues, cells, or extracts that are used as the sample. A biological sample can be processed differently depending on the assay being employed. For example, when detecting any variant nucleic acid molecule that decreases STAB1 expression and/or activity, preliminary processing designed to isolate or enrich the biological sample for the variant nucleic acid molecule can be employed. A variety of techniques may be used for this purpose. When detecting the level of any variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting a variant nucleic acid molecule that decreases STAB1 expression and/or activity, or the complement thereof. The methods comprise assaying a biological sample obtained from the subject to determine whether a nucleic acid molecule in the biological sample is a variant nucleic acid molecule that decreases STAB1 expression and/or activity.

In some embodiments, the variant nucleic acid molecule that decreases STAB1 expression and/or activity, or the complement thereof, is a genomic nucleic acid molecule having a nucleotide sequence comprising a guanine at a position corresponding to position 501 according to SEQ ID NO:17, or the complement thereof. In some embodiments, the variant nucleic acid molecule that decreases STAB1 expression and/or activity has a nucleotide sequence comprising a guanine at a position corresponding to position 501 according to SEQ ID NO:17.

In some embodiments, the biological sample comprises a cell or cell lysate. Such methods can further comprise, for example, obtaining a biological sample from the subject comprising a genomic nucleic acid molecule or mRNA molecule, and if mRNA, optionally reverse transcribing the mRNA into cDNA. Such assays can comprise, for example determining the identity of these positions of the particular nucleic acid molecule. In some embodiments, the method is an in vitro method.

In some embodiments, the determining step, detecting step, or sequence analysis comprises sequencing at least a portion of the nucleotide sequence of the variant nucleic acid molecule that decreases STAB1 expression and/or activity in the biological sample, wherein the sequenced portion comprises one or more variations that cause or result in decreased expression and/or activity of STAB1.

In some embodiments, the determining step, detecting step, or sequence analysis comprises sequencing at least a portion of the nucleotide sequence of the genomic nucleic acid molecule in the biological sample, wherein the sequenced portion comprises a position corresponding to position 501 according to SEQ ID NO:17, or the complement thereof. When the sequenced portion of the nucleic acid molecule in the biological sample comprises a guanine at a position corresponding to position 501 according to SEQ ID NO:17, then the nucleic acid molecule in the biological sample is a variant genomic nucleic acid molecule that decreases STAB1 expression and/or activity.

In some embodiments, the determining step, detecting step, or sequence analysis comprises: a) contacting the biological sample with a primer hybridizing to a portion of the nucleotide sequence of the genomic nucleic acid molecule, or the complement thereof, that is proximate to a position corresponding to position 501 according to SEQ ID NO:17, or the complement thereof; b) extending the primer at least through the position of the nucleotide sequence of the STAB1 genomic nucleic acid molecule, or the complement thereof, corresponding to position 501 according to SEQ ID NO:17, or the complement thereof; and c) determining whether the extension product of the primer comprises a guanine at a position corresponding to position 501 according to SEQ ID NO:17, or the complement thereof.

In some embodiments, the entire nucleic acid molecule is sequenced. In some embodiments, only a genomic nucleic acid molecule is analyzed. In some embodiments, only an mRNA is analyzed. In some embodiments, only a cDNA obtained from an mRNA is analyzed.

In some embodiments, the determining step, detecting step, or sequence analysis comprises: a) amplifying at least a portion of the nucleic acid molecule, or the complement thereof, in the biological sample, wherein the amplified portion comprises a guanine at a position corresponding to position 501 according to SEQ ID NO:17, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to position 501 according to SEQ ID NO:17, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the genomic nucleic acid molecule, or the complement thereof, comprising a guanine at a position corresponding to position 501 according to SEQ ID NO:17, or the complement thereof; and detecting the detectable label.

In some embodiments, the nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to a variant genomic sequence, variant mRNA sequence, or variant cDNA sequence and not the corresponding reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a variant nucleic acid molecule that decreases STAB1 expression and/or activity, or complement thereof, within a biological sample comprises a nucleotide sequence comprising a guanine at a position corresponding to position 501 according to SEQ ID NO:17, the biological sample can be subjected to an amplification method using a primer pair that includes a first primer derived from the 5′ flanking sequence adjacent to a guanine at a position corresponding to position 501 according to SEQ ID NO:17, and a second primer derived from the 3′ flanking sequence adjacent to a guanine at a position corresponding to position 501 according to SEQ ID NO:17 to produce an amplicon that is indicative of the presence of the SNP at positions encoding a guanine at a position corresponding to position 501 according to SEQ ID NO:17. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 501 according to SEQ ID NO:17 and at least 1, 2, 3, 4, 5, 6, 7, 8, 9, 10, or more nucleotides on each side of positions comprising a guanine at a position corresponding to position 501 according to SEQ ID NO:17.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a STAB1 polypeptide, or level or activity thereof, comprising performing an assay on a biological sample obtained from the subject to determine whether a STAB1 polypeptide in the biological sample is expressed at a decreased level or a decreased activity.

In some embodiments, the methods comprise performing an assay on a biological sample obtained from a subject to determine whether a STAB1 polypeptide in the biological sample comprises decreased expression and/or activity. In some embodiments, the detecting step comprises sequencing at least a portion of the STAB1 polypeptide to determine whether a STAB1 polypeptide in the biological sample comprises a predicted loss-of-function polypeptide. In some embodiments, the detecting step comprises an immunoassay for detecting the presence of a STAB1 polypeptide that comprises a predicted loss-of-function polypeptide.

In some embodiments, when the subject does not have a decreased expression and/or activity of a STAB1 polypeptide, the subject has an increased risk of developing a psychiatric disorder and/or a psychiatric disorder-associated MRI phenotype, such as schizophrenia, bipolar type II disorder, cognitive impairment, or an autism spectrum disorder. In some embodiments, when the subject has a decreased expression and/or activity of a STAB1 polypeptide, the subject has a decreased risk of developing a psychiatric disorder and/or a psychiatric disorder-associated MRI phenotype, such as schizophrenia, bipolar type II disorder, cognitive impairment, or an autism spectrum disorder.

The present disclosure also provides isolated nucleic acid molecules that hybridize to a variant nucleic acid molecule that decreases STAB1 expression and/or activity. In some embodiments, such isolated nucleic acid molecules hybridize to variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of a genomic nucleic acid molecule that includes a position corresponding to position 501 according to SEQ ID NO:17.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to a variant nucleic acid molecule that decreases STAB1 expression and/or activity. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of a variant nucleic acid molecule that decreases STAB1 expression and/or activity, or the complement thereof. In some embodiments, the portion comprises a position corresponding to position 501 according to SEQ ID NO:17, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any variant nucleic acid molecule that decreases STAB1 expression and/or activity. The primers described herein can be used to amplify the variant nucleic acid molecules, or a fragment thereof.

The present disclosure also provides pairs of primers comprising any of the primers described above. For example, if one of the primers' 3′-ends hybridizes to an adenine at a position corresponding to position 501 according to SEQ ID NO:16 (rather than a guanine) in a particular STAB1 nucleic acid molecule, then the presence of the amplified fragment would indicate the presence of a reference genomic nucleic acid molecule. Conversely, if one of the primers' 3′-ends hybridizes to a guanine at a position corresponding to position 501 according to SEQ ID NO:17 (rather than an adenine) in a particular nucleic acid molecule, then the presence of the amplified fragment would indicate the presence of a variant nucleic acid molecule that decreases STAB1 expression and/or activity. In some embodiments, the nucleotide of the primer complementary to the guanine at a position corresponding to position 501 according to SEQ ID NO:17 can be at the 3′ end of the primer.

In the context of the present disclosure “specifically hybridizes” means that the probe or primer (such as, for example, the alteration-specific probe or alteration-specific primer) does not hybridize to a nucleic acid sequence encoding a reference nucleic acid molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

In some embodiments, any of the methods described herein can further comprise determining a subject's gene burden of having a variant nucleic acid molecule that decreases STAB1 expression and/or activity that is associated with a decreased risk of developing a psychiatric disorder and/or a psychiatric disorder-associated MRI phenotype. The gene burden is the aggregate of all variant nucleic acid molecules that decrease STAB1 expression and/or activity, which can be carried out in an association analysis with psychiatric disorders and/or psychiatric disorder-associated MRI phenotypes. In some embodiments, the subject is homozygous for one or more variant nucleic acid molecules associated with a decreased risk of developing a psychiatric disorder and/or a psychiatric disorder-associated MRI phenotype. In some embodiments, the subject is heterozygous for one or more variant nucleic acid molecules associated with a decreased risk of a developing psychiatric disorder and/or a psychiatric disorder-associated MRI phenotype. The result of the association analysis suggests that variant nucleic acid molecules that decrease STAB1 expression and/or activity are associated with decreased risk of developing a psychiatric disorder and/or a psychiatric disorder-associated MRI phenotype. When the subject has a lower gene burden, the subject is at a higher risk of developing a psychiatric disorder and/or a psychiatric disorder-associated MRI phenotype and the subject is administered or continued to be administered the therapeutic agent that treats, prevents, or inhibits the psychiatric disorder and/or the psychiatric disorder-associated MRI phenotype in a standard dosage amount, and/or a STAB1 inhibitor. When the subject has a greater gene burden, the subject is at a decreased risk of developing a psychiatric disorder and/or a psychiatric disorder-associated MRI phenotype and the subject is administered or continued to be administered the therapeutic agent that treats, prevents, or inhibits the psychiatric disorder and/or the psychiatric disorder-associated MRI phenotype in an amount that is the same as or less than the standard dosage amount. The greater the gene burden, the lower the risk of developing a psychiatric disorder and/or a psychiatric disorder-associated MRI phenotype. Representative variant nucleic acid molecules that decrease STAB1 expression and/or activity include, but are not limited to, loss-of-function variants (e.g., stop gain, stop lost, start gain, start lost, splice donor, splice acceptor, frameshift insertion, and frameshift deletion) and missense variants.

In some embodiments, the subject's gene burden of having any one or more variant nucleic acid molecules that decrease STAB1 expression and/or activity represents a weighted aggregate of a plurality of the variant nucleic acid molecules. In some embodiments, the gene burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the STAB1 gene where the gene burden is the number of alleles multiplied by the association estimate with psychiatric disorders and/or psychiatric disorder-associated MRI phenotypes or related outcome for each allele (e.g., a weighted burden score). This can include any genetic variants, regardless of their genomic annotation, in proximity to the STAB1 gene (up to 10 Mb around the gene) that show a non-zero association with a psychiatric disorder and/or a psychiatric disorder-associated MRI phenotype-related trait in a genetic association analysis. In some embodiments, when the subject has a gene burden above a desired threshold score, the subject has a decreased risk of developing a psychiatric disorders and/or a psychiatric disorder-associated MRI phenotype. In some embodiments, when the subject has a gene burden below a desired threshold score, the subject has an increased risk of developing a psychiatric disorder and/or a psychiatric disorder-associated MRI phenotype.

In some embodiments, the gene burden may be divided into quintiles, e.g., top quintile, intermediate quintile, and bottom quintile, wherein the top quintile of gene burden corresponds to the lowest risk group and the bottom quintile of gene burden corresponds to the highest risk group. In some embodiments, a subject having a greater gene burden comprises the highest weighted gene burdens, including, but not limited to the top 10%, top 20%, top 30%, top 40%, or top 50% of gene burdens from a subject population. In some embodiments, the genetic variants comprise the genetic variants having association with psychiatric disorders and/or psychiatric disorder-associated MRI phenotypes in the top 10%, top 20%, top 30%, top 40%, or top 50% of p-value range for the association. In some embodiments, each of the identified genetic variants comprise the genetic variants having association with psychiatric disorders and/or psychiatric disorder-associated MRI phenotypes with p-value of no more than about 10-2, about 10-3, about 10-4, about 10-5, about 10-6, about 10-7, about 10-8, about 10-9, about 10-10, about 10-11, about 10-12, about 10-13, about 10-14, about or 10-15. In some embodiments, the identified genetic variants comprise the genetic variants having association with psychiatric disorders and/or psychiatric disorder-associated MRI phenotypes with p-value of less than 5×10-8. In some embodiments, the identified genetic variants comprise genetic variants having association with psychiatric disorders and/or psychiatric disorder-associated MRI phenotypes in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having gene burdens in the bottom decile, quintile, or tertile in a reference population. The threshold of the gene burden is determined on the basis of the nature of the intended practical application and the risk difference that would be considered meaningful for that practical application.

In some embodiments, when a subject is identified as having an increased risk of developing a psychiatric disorder and/or a psychiatric disorder-associated MRI phenotype, the subject is further administered a therapeutic agent that treats, prevents, or inhibits a psychiatric disorder and/or a psychiatric disorder-associated MRI phenotype, and/or a STAB1 inhibitor, as described herein. For example, when the subject does not have a variant nucleic acid molecule that decreases STAB1 expression and/or activity, and therefore has an increased risk of developing a psychiatric disorder and/or a psychiatric disorder-associated MRI phenotype, the subject is administered a STAB1 inhibitor. In some embodiments, such a subject is also administered a therapeutic agent that treats, prevents, or inhibits a psychiatric disorder and/or a psychiatric disorder-associated MRI phenotype. In some embodiments, when the subject is heterozygous for a variant nucleic acid molecule that decreases STAB1 expression and/or activity, the subject is administered the therapeutic agent that treats, prevents, or inhibits the psychiatric disorder and/or the psychiatric disorder-associated MRI phenotype in a dosage amount that is the same as or less than a standard dosage amount, and is also administered a STAB1 inhibitor. In some embodiments, the subject does not have a variant nucleic acid molecule that decreases STAB1 expression and/or activity. In some embodiments, the subject is heterozygous for a variant nucleic acid molecule that decreases STAB1 expression and/or activity. Furthermore, when the subject has a lower gene burden for having a variant nucleic acid molecule that decreases STAB1 expression and/or activity, and therefore has an increased risk of developing a psychiatric disorder and/or a psychiatric disorder-associated MRI phenotype, the subject is administered a therapeutic agent that treats, prevents, or inhibits the psychiatric disorder and/or the psychiatric disorder-associated MRI phenotype. In some embodiments, when the subject has a lower gene burden for having a variant nucleic acid molecule that decreases STAB1 expression and/or activity, the subject is administered the therapeutic agent that treats, prevents, or inhibits the psychiatric disorder and/or the psychiatric disorder-associated MRI phenotype in a dosage amount that is the same as or greater than the standard dosage amount administered to a subject who has a greater gene burden for having a variant nucleic acid molecule that decreases STAB1 expression and/or activity.

The nucleotide sequence of a reference STAB1 genomic nucleic acid molecule is set forth in SEQ ID NO:1. The nucleotide sequence of the genomic locus of the rs11921116 SNP encompassing chromosome 3 positions 52,473,390-52,474,390 according to GRCh38/hg38 human genome assembly is set forth in SEQ ID NO:16 (reference sequence). Referring to SEQ ID NO:16, position 501 is an adenine. In the variant rs11921116 SNP allele, the adenine at position 501 is replaced with a guanine. The nucleotide sequence of this variant genomic nucleic acid locus is set forth in SEQ ID NO:17.

The nucleotide sequence of a STAB1 reference mRNA molecule is set forth in SEQ ID NO:2. The nucleotide sequence of another STAB1 reference mRNA molecule is set forth in SEQ ID NO:3. The nucleotide sequence of another STAB1 reference mRNA molecule is set forth in SEQ ID NO:4. The nucleotide sequence of another STAB1 reference mRNA molecule is set forth in SEQ ID NO:5. The nucleotide sequence of another STAB1 reference mRNA molecule is set forth in SEQ ID NO:6. The nucleotide sequence of another STAB1 reference mRNA molecule is set forth in SEQ ID NO:7.

The nucleotide sequence of a STAB1 reference cDNA molecule is set forth in SEQ ID NO:8. The nucleotide sequence of another STAB1 reference cDNA molecule is set forth in SEQ ID NO:9. The nucleotide sequence of another STAB1 reference cDNA molecule is set forth in SEQ ID NO:10. The nucleotide sequence of another STAB1 reference cDNA molecule is set forth in SEQ ID NO:11. The nucleotide sequence of another STAB1 reference cDNA molecule is set forth in SEQ ID NO:12. The nucleotide sequence of another STAB1 reference cDNA molecule is set forth in SEQ ID NO:13.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:16, SEQ ID NO:2, or SEQ ID NO:8). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence, wherein the nucleotide sequence comprises a guanine at a position corresponding to position 501 according to SEQ ID NO:17 means that if the nucleotide sequence of the genomic nucleic acid molecule is aligned to the sequence of SEQ ID NO:17, the sequence has a guanine residue at the position that corresponds to position 501 of SEQ ID NO:17. In other words, these phrases refer to a nucleic acid molecule, wherein the genomic nucleic acid molecule has a nucleotide sequence that comprises a guanine residue that is homologous to the guanine residue at position 501 of SEQ ID NO:17.

As described herein, a position within a genomic nucleic acid molecule that corresponds to position 501 according to SEQ ID NO:17, for example, can be identified by performing a sequence alignment between the nucleotide sequence of a particular nucleic acid molecule and the nucleotide sequence of SEQ ID NO:17. A variety of computational algorithms exist that can be used for performing a sequence alignment to identify a nucleotide position that corresponds to, for example, position 501 in SEQ ID NO:17. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of STAB1 reference polypeptides are set forth in SEQ ID NO:14 (Isoform 1), and SEQ ID NO:15 (Isoform 2).

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit a psychiatric disorder and/or a psychiatric disorder-associated MRI phenotype for use in the treatment of a psychiatric disorder and/or a psychiatric disorder-associated MRI phenotype (or for use in the preparation of a medicament for treating a psychiatric disorder and/or a psychiatric disorder-associated MRI phenotype) in a subject, wherein the subject has any of the variant nucleic acid molecule that decrease STAB1 expression and/or activity described herein. The therapeutic agents that treat or inhibit the psychiatric disorder and/or the psychiatric disorder-associated MRI phenotype can be any of the therapeutic agents that treat or inhibit the psychiatric disorders and/or the psychiatric disorder-associated MRI phenotypes described herein.

In some embodiments, the subject is identified as having a variant genomic nucleic acid molecule, wherein the variant genomic nucleic acid molecule has a nucleotide sequence comprising a guanine at a position corresponding to position 501 according to SEQ ID NO:17, or the complement thereof.

The present disclosure also provides STAB1 inhibitors for use in the treatment of a psychiatric disorder and/or a psychiatric disorder-associated MRI phenotype (or for use in the preparation of a medicament for treating a psychiatric disorder and/or a psychiatric disorder-associated MRI phenotype) in a subject, wherein the subject is heterozygous for a variant nucleic acid molecule that decreases STAB1 expression and/or activity, or wherein the subject does not have a variant nucleic acid molecule that decreases STAB1 expression and/or activity. The STAB1 inhibitors can be any of the STAB1 inhibitors described herein.

In some embodiments, the subject does not have a variant nucleic acid molecule that decreases STAB1 expression and/or activity.

In some embodiments, the subject is identified as being heterozygous for a variant nucleic acid molecule that decreases STAB1 expression and/or activity, wherein the variant nucleic acid molecule has a nucleotide sequence comprising a guanine at a position corresponding to position 501 according to SEQ ID NO:17, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Predicted Loss of Function Variants in STAB1 have a Protective Effect on Brain MRI Changes

STAB1 burden masks show protective associations with multiple MRI phenotypes (specifically, increase in volume of grey matter in both left and right putamen, and in both left and right ventral striatum) in the UKB 450 exome analysis. Among the 70 genes reported to be associated with MRI traits, STAB1 showed the strongest as well as the largest number of associations (data not shown). For example, a burden of pLOF with MAF <0.0001 in STAB1 is associated with higher median T2star in left putamen (Beta=1, Cl=0.76-1.3, P=5.5e-13) and right putamen (Beta=1, Cl=0.76-1.3, P=7.1e-13). This suggests that STAB1 inhibition will decrease the risk for age associated loss of brain structural integrity.

### Example 2: Predicted Loss of Function Variants in STAB1 have a Protective Effect on Cognitive Function

STAB1 M1 burden masks show protective associations with a fluid intelligence score in the UKB, although with nominal statistical significance (Table 2). The effect size was largest for the M1 mask with an AAF<0.0001% (Beta=0.13, Cl=0.01-0.25, P=0.03), which then reduces as the AAF increases as shown in Table 2 suggesting that the higher the deleteriousness, the stronger the association with the fluid intelligence. Hence, the results suggest that STAB1 inhibition may be protective against cognitive impairment.

TABLE 2
M1 associations with fluid intelligence score in the UK Biobank

| Mask | Beta | P | Sample counts |
|---|---|---|---|
| M1 (AAF < 0.001%) | 0.13 (0.01-0.25) | 0.03 | 172334\|215\|0 |
| M1 (AAF < 0.01%) | 0.078 (−0.0004-0.15) | 0.05 | 172027\|522\|0 |
| M1 (AAF < 0.1%) | 0.10 (0.04-0.16) | 0.001 | 171719\|829\|1 |
| M1 (AAF < 1%) | 0.04 (−0.010-0.09) | 0.12 | 171284\|1264\|1 |

### Example 3: Common Variants Around STAB1 are Strongly Associated with Bipolar Disorder and Schizophrenia

STAB1 is located within a classic psychiatric GWAS locus. The GWAS region near STAB1 has been first reported in 2011 in a GWAS of bipolar disorder and was one of the GWAS loci discovered in every subsequent GWAS of bipolar disorder. Locus zoom plots of STAB1 locus based on the latest GWAS of bipolar disorder from the psychiatric genomics consortium (PGC), based on the latest GWAS of bipolar type 1 disorder from the PGC, based on the latest GWAS of bipolar type II disorder from the PGC are summarized in Table 3 (SNP=r52577831; PMID=34002096). The same locus is also strongly associated with schizophrenia (Table 4; SNP=r52710323; PMID=MedRxiv) and many other psychiatric disorders.

TABLE 3
GWAS locus near STAB1 associated with bipolar disorder

| Phenotype | OR | P value | N case \|N control |
|---|---|---|---|
| BPD | 1.070 | 3.8e−13 | 41917\|371549 |
| BPD Type 1 | 1.066 | 2.4e−08 | 25060\|449978 |
| BPD Type II | 1.078 | 9.1e−05 | 6781\|364075 |

TABLE 4
GWAS locus near STAB1 associated with schizophrenia

| Phenotype | OR | P value | N case\|N control |
|---|---|---|---|
| Schizophrenia | 1.077 | 5.9e−22 | 67390\|94015 |

Finally, mendelian randomization IVW analysis shows that increased STAB1 expression is causally associated with increased risk for schizophrenia, Bipolar II (manic type) but not with Bipolar I, suggesting blocking STAB1 will be beneficial to treatment of schizophrenia and bipolar II disorder (Table 5).

TABLE 5
Mendelian Randomization IVW analysis (exposure =
STAB1 blood eQTLs, n = 20)

| Psych | Beta | SE | P value |
|---|---|---|---|
| Schizophrenia | 0.116 | 0.03 | 0.0001 |
| Bipolar | 0.104 | 0.029 | 0.003 |
| Bipolar I | 0.044 | 0.041 | 0.28 |
| Bipolar II | 0.206 | 0.038 | 1.05e−07 |

### Example 4: STAB1 Gene Burden Associations with Depression and Other Psychiatric Traits

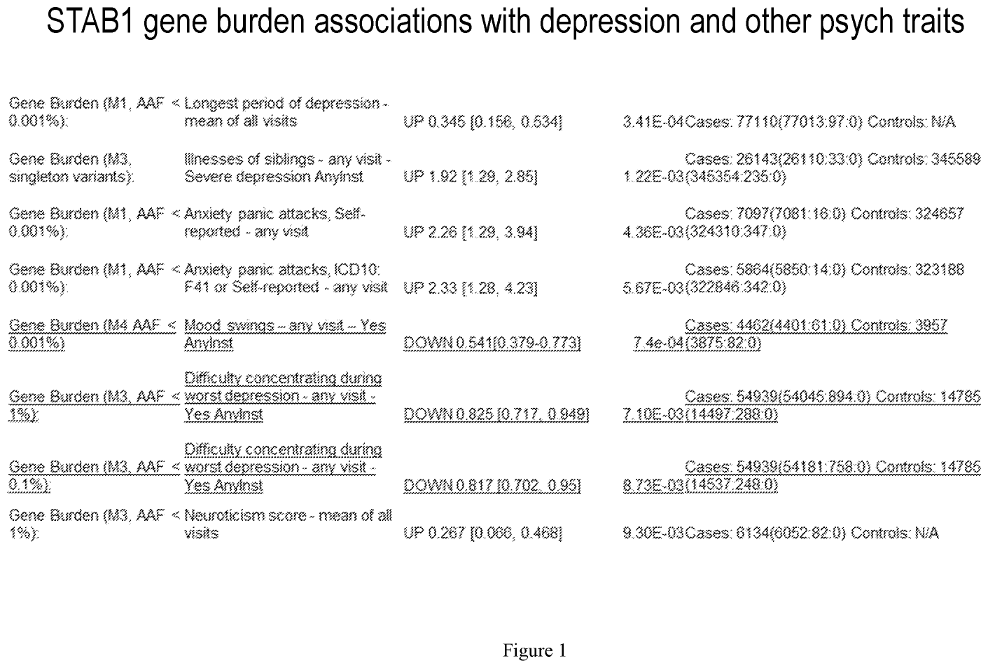

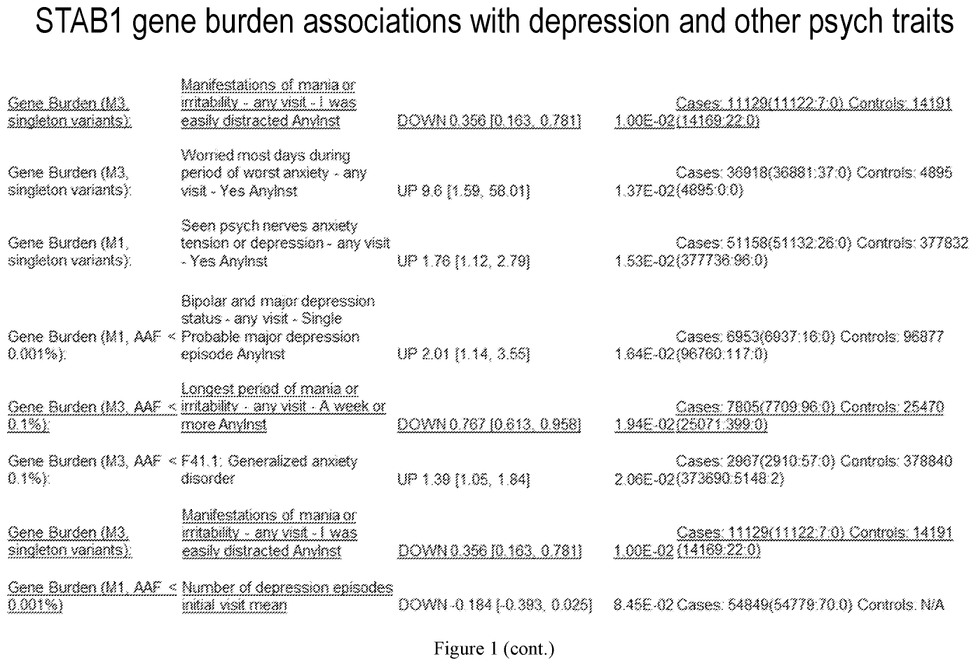

FIG. 1 shows STAB1 gene burden associations with depression and other psychiatric traits. The gene burden associations of STAB1 with various psychiatric phenotypes were extracted from an internal browser that contains results from high throughput GWAS and ExWAS analysis of all phenotypes in UK Biobank and GHS cohorts.

### Example 5: A Common Variant Near STAB1 is Associated with Decreased STAB1 Expression in Blood, Better MRI Outcomes, and Decreased Risk for Multiple Major Psychiatric Disorders

A search was focused on finding common variants that show association with STAB1 expression in blood as well as with MRI and psychiatric phenotypes. A common variant (r511921116) was identified as having a significant cis eQTL for STAB1 in blood based on the largest blood eQTL study available to date. The minor allele of rs11921116 is G (AF=˜5%) and the major allele is A (AF=˜95%). The G allele is associated with decreased STAB1 expression in blood (Z=−36.21, P=0) based on an analysis of 29,863 individuals. The SNP is eQTL for multiple nearby genes, however, the strongest association and largest effect size was observed for STAB1.

The G allele of rs11921116 was associated with increased median T2star in the right and left putamen, pallidum and caudate brain regions in the RGC analysis of the UK Biobank MRI phenotypes. The associations are shown in Table 6.

TABLE 6
Association of rs11921116 with Median T2star MRI
phenotypes in the UKB

| Median T2star in | Beta | P value | Sample size |
|---|---|---|---|
| Putamen right | 0.127 | 1.6e−15 | 32,277 |
| Putamen left | 0.117 | 2.2e−13 | 32,277 |
| Caudate right | 0.107 | 2.1e−11 | 32,277 |
| Caudate left | 0.096 | 1.9e−9 | 32,277 |
| Pallidum left | 0.078 | 7.6e−7 | 32,277 |
| Pallidum right | 0.075 | 1.8e−6 | 32,277 |

The G allele of rs11921116 was associated with a decreased risk for multiple psychiatric disorders based on the publicly available GWAS summary statistics from the PGC. The associations are shown in Table 7. The SNP was associated with only bipolar disorder type II, but not with bipolar disorder type I. In addition, the largest protective effect size (OR=0.88) was observed for bipolar type II. The results suggest that STAB1 inhibition may be a therapeutic option to decrease the risk of developing bipolar disorder type II or to treat bipolar disorder type II.

TABLE 7
Association of rs11921116 with decreased risk for multiple
psychiatric disorders in the PGC

| Phenotype | OR | P value | Study | PMID |
|---|---|---|---|---|
| Schizophrenia | 0.94 | 0.001 | PGC3 2020 | MedRxiv |
| Bipolar disorder | 0.94 | 0.006 | Mullins et al 2021 | 34002096 |
| Bipolar type 1 | 0.98 | 0.47 | Mullins et al 2021 | 34002096 |
| Bipolar type II | 0.88 | 0.01 | Mullins et al 2021 | 34002096 |
| Major | 0.98 | 0.25 | Wray et al 2018 | 29700475 |
| depression | | | | |
| ADHD | 0.95 | 0.08 | Demontis et al | 29700475 |
| | | | 2019 | |
| ASD | 0.94 | 0.04 | Grove et al 2019 | 30804558 |
| Brain volume | 0.027* | 0.057 | Jansen et al 2020 | 33154357 |

There exists a consistent protective effect size for all disorders and the association of the same SNP rs11921116 with MRI phenotypes and STAB1 expression in blood.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a psychiatric disorder and/or a psychiatric disorder-associated MRI phenotype, or having schizophrenia, or having bipolar type II disorder, or having cognitive impairment, or having an autism spectrum disorder (ASD), the method comprising administering a Stabilin 1 (STAB1) inhibitor to the subject.

2-5. (canceled)

6. The method according to claim 1, wherein the STAB1 inhibitor comprises an inhibitory nucleic acid molecule.

7. The method according to claim 6, wherein the inhibitory nucleic acid molecule comprises an antisense nucleic acid molecule, a small interfering RNA (siRNA), or a short hairpin RNA (shRNA) that hybridizes to a STAB1 nucleic acid molecule.

8-14. (canceled)

15. The method according to claim 1, further comprising detecting the presence or absence of a variant nucleic acid molecule that decreases expression and/or activity of STAB1 in a biological sample obtained from the subject.

16. The method according to claim 15, further comprising administering a therapeutic agent that treats or inhibits a psychiatric disorder and/or a psychiatric disorder-associated MRI phenotype in a standard dosage amount to a subject wherein the variant nucleic acid molecule is absent from the biological sample.

17. The method according to claim 15, further comprising administering a therapeutic agent that treats or inhibits a psychiatric disorder and/or a psychiatric disorder-associated MRI phenotype in a dosage amount that is the same as or less than a standard dosage amount to a subject that is heterozygous for the variant nucleic acid molecule.

18. The method according to claim 15, wherein the variant nucleic acid molecule is a genomic nucleic acid molecule having a nucleotide sequence comprising a guanine at a position corresponding to position 501 according to SEQ ID NO:17.

19. (canceled)

20. The method according to claim 15, wherein the detecting step comprises sequencing at least a portion of the nucleotide sequence of the genomic nucleic acid molecule, or the complement thereof, in the biological sample, wherein the sequenced portion comprises a position corresponding to position 501 according to SEQ ID NO:17, or the complement thereof;
wherein when the sequenced portion of the genomic nucleic acid molecule in the biological sample comprises a guanine at a position corresponding to position 501 according to SEQ ID NO:17, then the genomic nucleic acid molecule in the biological sample is a variant genomic nucleic acid molecule that decreases expression and/or activity of STAB 1.

21. The method according to claim 15, wherein the detecting step comprises:
a) contacting the biological sample with a primer hybridizing to a portion of the nucleotide sequence of the genomic nucleic acid molecule, or complement thereof, that is proximate to a position corresponding to position 501 according to SEQ ID NO:17,
b) extending the primer at least through the position of the nucleotide sequence of the genomic nucleic acid molecule, or complement thereof, corresponding to position 501 according to SEQ ID NO:17; and
c) determining whether the extension product of the primer comprises a guanine at a position corresponding to position 501 according to SEQ ID NO:17.

22. The method according to claim 15, wherein the detecting step comprises:
a) amplifying at least a portion of the genomic nucleic acid molecule, or complement thereof, in the biological sample, wherein the portion comprises a guanine at a position corresponding to position 501 according to SEQ ID NO:17, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 501 according to SEQ ID NO:17, or the complement thereof; and
d) detecting the detectable label.

23. The method according to claim 15, wherein the detecting step comprises:
contacting the genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the genomic nucleic acid molecule, or the complement thereof, comprising a guanine at a position corresponding to position 501 according to SEQ ID NO:17, or the complement thereof; and
detecting the detectable label.

24. A method of treating a subject with a therapeutic agent that treats or inhibits a psychiatric disorder and/or a psychiatric disorder-associated MRI phenotype, wherein the subject has a psychiatric disorder and/or a psychiatric disorder-associated MRI phenotype, the method comprising:
determining whether the subject has a variant nucleic acid molecule that decreases expression and/or activity of STAB1 by:
obtaining or having obtained a biological sample from the subject; and
performing or having performed a sequence analysis on the biological sample to determine if the subject has a genotype comprising the variant nucleic acid molecule that decreases expression and/or activity of STAB 1; and
administering or continuing to administer the therapeutic agent that treats or inhibits the psychiatric disorder and/or the psychiatric disorder-associated MRI phenotype in a standard dosage amount to a subject that does not have the variant nucleic acid molecule that decreases expression and/or activity of STAB1, and administering a STAB1 inhibitor to the subject; and
administering or continuing to administer the therapeutic agent that treats or inhibits the psychiatric disorder and/or the psychiatric disorder-associated MRI phenotype in an amount that is the same as or less than a standard dosage amount to a subject that is heterozygous for the variant nucleic acid molecule that decreases expression and/or activity of STAB1, and administering a STAB1 inhibitor to the subject;
wherein the presence of a genotype having the variant nucleic acid molecule that decreases expression and/or activity of STAB1 indicates the subject has a reduced risk of developing a psychiatric disorder and/or a psychiatric disorder-associated MRI phenotype.

25. The method according to claim 24, wherein the subject does not have the variant nucleic acid molecule that decreases expression and/or activity of STAB1, and the subject is administered or continued to be administered the therapeutic agent that treats or inhibits the psychiatric disorder and/or the psychiatric disorder-associated MRI phenotype in a standard dosage amount, and is administered a STAB1 inhibitor.

26. The method according to claim 24, wherein the subject is heterozygous for the variant nucleic acid molecule that decreases expression and/or activity of STAB1, and the subject is administered or continued to be administered the therapeutic agent that treats or inhibits the psychiatric disorder and/or the psychiatric disorder-associated MRI phenotype in an amount that is the same as or less than a standard dosage amount, and is administered a STAB1 inhibitor.

27. The method according to claim 24, wherein the variant nucleic acid molecule is a genomic nucleic acid molecule having a nucleotide sequence comprising a guanine at a position corresponding to position 501 according to SEQ ID NO:17.

28. The method according to claim 24, wherein the sequence analysis comprises sequencing at least a portion of the nucleotide sequence of the genomic nucleic acid molecule, or the complement thereof, in the biological sample, wherein the sequenced portion comprises a position corresponding to position 501 according to SEQ ID NO:17, or the complement thereof;
wherein when the sequenced portion of the genomic nucleic acid molecule, or the complement thereof, in the biological sample comprises a guanine at a position corresponding to position 501 according to SEQ ID NO:17, then the genomic nucleic acid molecule in the biological sample is a variant genomic nucleic acid molecule that decreases expression and/or activity of STAB 1.

29. The method according to claim 24, wherein the sequence analysis comprises:
a) contacting the biological sample with a primer hybridizing to a portion of the nucleotide sequence of the genomic nucleic acid molecule, or the complement thereof, that is proximate to a position corresponding to position 501 according to SEQ ID NO:17;
b) extending the primer at least through the position of the nucleotide sequence of the genomic nucleic acid molecule, or the complement thereof, corresponding to position 501 according to SEQ ID NO:17; and
c) determining whether the extension product of the primer comprises a guanine at a position corresponding to position 501 according to SEQ ID NO:17.

30. (canceled)

31. The method according to claim 24, wherein the sequence analysis comprises:
a) amplifying at least a portion of the genomic nucleic acid molecule, or the complement thereof, in the biological sample, wherein the portion comprises a guanine at a position corresponding to position 501 according to SEQ ID NO:17, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to position 501 according to SEQ ID NO:17, or the complement thereof; and
d) detecting the detectable label.

32. The method according to claim 24, wherein the sequence analysis comprises:
contacting the genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the genomic nucleic acid molecule, or the complement thereof, comprising a guanine at a position corresponding to position 501 according to SEQ ID NO:17, or the complement thereof; and
detecting the detectable label.

33. The method according to claim 24, wherein the nucleic acid molecule is present within a cell obtained from the subject.

34. The method according to claim 24, wherein the STAB1 inhibitor comprises an inhibitory nucleic acid molecule.

35. The method according to claim 34, wherein the inhibitory nucleic acid molecule comprises an antisense nucleic acid molecule, a small interfering RNA (siRNA), or a short hairpin RNA (shRNA) that hybridizes to a STAB1 nucleic acid molecule.

36-63. (canceled)
